(19) United States
(12) Patent Application Publication
(10) Pub. No.: US 2026/0096842 A1
(43) Pub. Date: Apr. 9, 2026

(54) Balloon Catheter Ablation Systems and Methods for Patterned Tissue Ablation

(71) Applicant: Virender K. Sharma, Paradise Valley, AZ (US)

(72) Inventor: Virender K. Sharma, Paradise Valley, AZ (US)

(21) Appl. No.: 19/349,912

(22) Filed: Oct. 3, 2025

Related U.S. Application Data
(60) Provisional application No. 63/703,817, filed on Oct. 4, 2024.
(60) Provisional application No. 63/741,921, filed on Jan. 5, 2025.

(51) Int. Cl. A61B 18/04; A61B 18/00; A61B 18/02

(52) U.S. Cl. CPC A61B 18/04; A61B 2018/00238; A61B 2018/00255; A61B 2018/00404; A61B 2018/00511; A61B 2018/00577; A61B 18/0218; A61B 2018/048

(57) ABSTRACT

Ablation catheters include an outer balloon positioned at a distal end of the catheter and configured to have an inner balloon disposed therein. A perforated layer intervenes between the outer balloon and the inner balloon. The outer balloon is inflated with a first fluid that has a temperature less than 100 degrees Celsius, while the inner balloon is inflated with heated vapor. An area of contact between the two balloons, comprising a surface area less than the total surface area of either balloon, creates an ablation zone comprising therapeutic and sub-therapeutic ablation zones that correspond to the pattern provided by the intervening perforated layer, for ablating target tissue through the transfer of thermal energy.

### CROSS-REFERENCE

The present application relies on U.S. Patent Provisional Application No. 63/741,921, titled “Methods and Devices for Endovascular Treatment” and filed on Jan. 5, 2025 and U.S. Patent Provisional Application No. 63/703,817, titled “Heated Vapor Ablation Systems and Methods” and filed on Oct. 4, 2024, for priority, all of which are incorporated herein by reference in their entirety.

### FIELD

The present specification relates to systems and methods configured to generate and deliver an ablative agent for ablation therapy. More particularly, the present specification relates to a catheter and vapor generation system for delivering thermal-based ablation therapy to a tissue. More specifically, the present specification relates to medical devices that deliver therapy by producing thermal effects via tissue heating and/or cooling.

### BACKGROUND

Ablation, as it pertains to the present specification, relates to the removal or destruction of a body tissue, via the introduction of a destructive agent, such as radiofrequency energy, electroporation, laser energy, ultrasonic energy, microwave energy, lasers, photoablation, sonication, cyroagents, hot fluid or steam. Ablation is commonly used to eliminate diseased or unwanted tissues, such as, but not limited to cysts, polyps, tumors, hemorrhoids, and other similar lesions including arrhythmia generating cardiovascular tissue, or eliminate certain entities of certain tissues, in particular electrical conductivity. Ablation is also used to ablate nerves in order to produce neurogenic effects including, but not limited to pain control, disrupting a neural pathway to treat hypertension, OAB, urinary or fecal incontinence

Hypertension is a condition in which pressure of blood in arteries is persistently elevated. Hypertension is conventionally a long-term condition. Some of the factors that contribute to high blood pressure include chronic kidney disease and narrowing of kidney arteries.

Radiofrequency, microwave, absolute alcohol or high intensity focus ultrasound (HIFU) ablation procedures are typically used to treat resistant hypertension, which is otherwise difficult to control through medication. In one of the procedures, nerves in the wall of renal artery are ablated to reduce sympathetic afferent and efferent activity to the kidney. Such procedures result in decrease in blood pressure. In an ablation procedure, an ablation device is directed to precise location(s) in the renal artery. These points are then isolated and destroyed. Pulmonary artery denervation (PADN) is an ablation procedure used to treat pulmonary hypertension (PH). The procedure targets nerves in the pulmonary artery trunk, disrupting sympathetic nerve activity to rebalance the autonomic nervous system. Denervation results in improved pulmonary function by reducing pulmonary artery pressure and vascular resistance.

Steam-based ablation systems, such as the ones disclosed in U.S. Pat. Nos. 9,615,875, 9,433,457, 9,376,497, 9,561,068, 9,561,067, and 9,561,066, disclose ablation systems that controllably deliver steam through one or more lumens toward a tissue target. One problem that all such steam-based ablation systems have is the potential overheating or burning of healthy tissue. Steam passing through a channel within a body cavity heats up surfaces of the channel and may cause exterior surfaces of the medical tool, other than the operational tool end itself, to become excessively hot. As a result, physicians may unintentionally burn healthy tissue when external portions of the device, other than the distal operational end of the tool, accidentally contacts healthy tissue. U.S. Pat. Nos. 9,561,068, 9,561,067, and 9,561,066 are hereby incorporated herein by reference.

Double balloon ablation catheters having inner and outer balloons, such as the ones disclosed in U.S. Pat. Nos. 7,727,228, 7,850,685, 8,425,456, and 8,679,104, maintain the outer balloon under a vacuum with almost no space between the inner and outer balloons. The purpose of such a construction is to provide a backup covering (the outer balloon) in case of a catastrophic failure of the inner balloon. Hence, during operation, the dimensions of the inner and outer balloons and shape of the inner and outer balloons are substantially the same and, during operation of the catheter, the outer balloon fits on the inner balloon like a glove.

Conventional double-balloon devices typically create a circumferential contact zone when an inner balloon, inflated with heated vapor or other therapeutic medium, expands against an outer balloon inflated with air or fluid. In such systems, the line or band of contact between the inner and outer balloons is generally continuous, forming an annulus of substantially uniform (within +/−10%) radial thickness. The uniformity of this annular contact often results in a circumferential band of uniform heating, which may not be desirable where selective ablation or patterned energy application is required.

What is needed is a catheter configured to concurrently direct ablative vapor heat toward a tissue, firmly position the catheter in the right tissue location, avoid burning healthy, or non-targeted tissue, including blood, and controllably deliver ablative energy to the target location. It would be further desirable to have hot fluid/steam-based ablation devices that integrate into the catheter safety mechanisms which prevent unwanted thermal injury to the patient and the operator during use. It is further desirable to be able to provide a way to increase, or augment, a natural cooling process to thereby decrease treatment time. It is also desirable to create dynamic or variable ablation (hot) and cooling zones that can vary with, and accommodate to, a patient's anatomy. It is desirable to provide an easy to implement heated ablation zone and cooling mechanism that does not rely on a separate medical tool to deliver fluid to cool the treatment area. What is also needed is systems and methods for producing non-circumferential, for example, spiral, thermal ablation wherein the ablation is provided to a desirable depth in a non-circumferential fashion in order to avoid complications, such as, stricture formation.

### SUMMARY

The following embodiments and aspects thereof are described and illustrated in conjunction with systems, tools and methods, which are meant to be exemplary and illustrative, and not limiting in scope. The present application discloses numerous embodiments.

In one aspect, the present disclosure provides a structure, namely an intermediate material layer, that modifies a continuous annular thermal contact into a non-continuous, discretized arrangement of thermal application sites. A patterned layer or material buffer positioned between an inner balloon and an outer balloon interrupts portions of the annular contact band, thereby converting the contact into a set of separated ablative regions distributed around the circumference.

The present specification discloses a method of ablating a target tissue, the method comprising: positioning a catheter proximate the target tissue of a patient, wherein the catheter comprises an elongate body having a lumen, a proximal end and a distal end and wherein an outer balloon, a perforated layer and an inner balloon are positioned at the distal end such that the inner balloon is positioned within the outer balloon and the perforated layer intervenes between the inner balloon and the outer balloon, wherein the perforated layer comprises a contiguous sheet with non-contiguous gaps; inflating the outer balloon with a first fluid to increase a pressure of the outer balloon to a first outer balloon pressure; expanding the perforated layer within the inflated outer balloon; and infusing a second fluid comprising an ablative agent into the inner balloon, wherein the second fluid/ablative agent is at least one of heated vapor, heated fluid, or cooling agents that use heating or cooling of tissue to produce a desired therapeutic effect, to increase a pressure of the inner balloon to a first inner balloon pressure, wherein infusing the second fluid into the inner balloon creates an ablation zone and wherein a surface area of the ablation zone is defined by a portion of the inner balloon contacting a portion of the outer balloon through the perforated layer to thereby allow for thermal transfer from the second fluid in the inner balloon through the ablation zone to the targeted tissue, wherein the ablation zone comprises a therapeutic ablation zone where a first portion of the inner balloon contacts a first portion of the outer balloon through the non-contiguous gaps of the perforated layer, and a sub-therapeutic zone where a second portion of the inner balloon contacts a second portion of the outer balloon through the contiguous sheet of the perforated layer.

Optionally, the target tissue is a renal artery, a portion of the renal artery, a renal vein, a portion of the renal vein or a nerve supplying any of these structures.

Optionally, the target tissue is a hepatic artery, a portion of the hepatic artery, a hepatic vein, a portion of the hepatic vein, or a nerve supplying any of these structures.

Optionally, the target tissue is a pulmonary artery, a portion of the pulmonary artery, a pulmonary vein, a portion of the pulmonary vein, or a nerve supplying any of these structures.

Optionally, the target tissue is a celiac artery, a portion of the celiac artery, a celiac vein, a portion of the celiac vein, or a nerve supplying any of these structures.

Optionally, the target tissue is an artery, a vein, or a nerve.

Optionally, the first pre-determined period of time is between 1 second and 5 minutes. Optionally, the first outer balloon pressure is maintained for the first pre-determined period of time.

Optionally, the catheter comprises a plurality of electrodes positioned proximate the distal end and wherein the ablative agent is generated by directing saline through the lumen and over the plurality of electrodes.

Optionally, the first fluid is air, CO₂, saline, water, a contrast agent, or a mixture of any of these.

Optionally, the ablative agent comprises steam and a temperature of the heated vapor is at least 100° C.

Optionally, the ablative agent comprises hot fluid and a temperature of the ablative agent is <100° C.

Optionally, the ablative agent is a cryogen, hot water, steam, or any one of these mixed with a contrast agent.

The present specification also discloses a system for ablating a target tissue, the system comprising: a catheter, wherein the catheter comprises: a proximal end; a distal end; an elongate body extending between the proximal end and the distal end; a first lumen; a second lumen; at least one heating chamber positioned within the second lumen and configured to generate heated fluid or heated vapor from a fluid; an outer balloon positioned at the distal end; an inner balloon, wherein the inner balloon is positioned within the outer balloon; and a perforated layer intervening between the inner balloon and the outer balloon, wherein the perforated layer comprises a contiguous sheet with non-contiguous gaps; an RF generator electrically coupled to the at least one heating chamber; a first fluid pump in fluid communication with the outer balloon through the first lumen; and a second fluid pump in fluid communication with the inner balloon through the second lumen; wherein infusing or generating heated fluid in the inner balloon creates a therapeutic ablation zone where a first portion of the inner balloon contacts a first portion of the outer balloon through the non-contiguous gaps of the perforated layer, and a sub-therapeutic ablation zone where a second portion of the inner balloon contacts a second portion of the outer balloon through the contiguous sheet of the perforated layer.

Optionally, the outer balloon comprises a plurality of electrodes attached to an outer surface of the outer balloon. Optionally, the plurality of electrodes are sensor electrodes to sense any of temperature, pressure, impedance or any signal that can be used to monitor or direct the treatment effect.

Optionally, the distal end comprises a plurality of infusion ports where the inner balloon is positioned. Optionally, the plurality of infusion ports provide an exit for heated vapor generated by the at least one heating chamber into the inner balloon.

Optionally, the at least one heating chamber comprises a plurality of electrodes positioned proximate the distal end and wherein the heated vapor is generated by directing saline through a second lumen in the elongate body and over the plurality of electrodes.

Optionally, the first lumen communicates air, CO₂, water, saline, or any one of these mixed with a contrast agent.

Optionally, the heated vapor comprises steam and a temperature of the heated vapor is at least 100° C.

The present specification also discloses a catheter for ablating a target tissue, the catheter comprising: an inflatable outer balloon at a distal end of the catheter; an expandable inner balloon configured to expand when heated vapor is infused within the inner balloon, wherein the inner balloon is positioned within the outer balloon and the inner balloon is coaxial with the outer balloon; and a perforated layer intervening between the inner balloon and the outer balloon, wherein the perforated layer comprises a contiguous sheet with non-contiguous gaps; wherein infusing heated vapor into the inner balloon creates a therapeutic ablation zone where a first portion of the inner balloon contacts a first portion of the outer balloon through the non-contiguous gaps of the perforated layer, and a sub-therapeutic ablation zone where a second portion of the inner balloon contacts a second portion of the outer balloon through the contiguous sheet of the perforated layer.

Optionally, the outer balloon is inflated using air or CO₂.

Optionally, the heated vapor comprises steam and a temperature of the heated vapor is at least 100° C.

Optionally, the inner balloon is smaller in size than the outer balloon by at least 10%.

Optionally, the inner balloon is movable along an axis of the inner balloon and the outer balloon.

The aforementioned and other embodiments of the present specification shall be described in greater depth in the drawings and detailed description provided below.

### BRIEF DESCRIPTION OF THE DRAWINGS

The accompanying drawings illustrate various embodiments of systems, methods, and embodiments of various other aspects of the disclosure. Any person with ordinary skills in the art will appreciate that the illustrated element boundaries (e.g. boxes, groups of boxes, or other shapes) in the figures represent one example of the boundaries. It may be that in some examples one element may be designed as multiple elements or that multiple elements may be designed as one element. In some examples, an element shown as an internal component of one element may be implemented as an external component in another and vice versa. Furthermore, elements may not be drawn to scale. Non-limiting and non-exhaustive descriptions are described with reference to the following drawings. The components in the figures are not necessarily to scale, emphasis instead being placed upon illustrating principles.

FIG. 1A illustrates an ablation system having a catheter with inner and outer balloons and an intervening layer between the two balloons, in accordance with an embodiment of the present specification;

FIG. 1B illustrates an ablation system having a catheter with inner and outer balloons and an intervening layer between the two balloons and a semi-porous thermal chamber within the inner balloon, in accordance with some embodiments of the present specification;

FIG. 1C illustrates a continuous annulus formed from an inflated inner balloon positioned within an inflated outer balloon with no intermediate layer or intervening layer positioned between;

FIG. 1D illustrates a non-continuous pattern created by positioning a selectively patterned intervening or intermediate layer between an inner balloon and an outer balloon, in accordance with some embodiments of the present specification;

FIG. 2A illustrates a sectional view of a catheter assembly of a device having a semi-porous thermal chamber, in accordance with some embodiments of the present specification;

FIG. 2B illustrates a cross-sectional view along an axis A-A of the catheter assembly shown in FIG. 2A;

FIG. 2C illustrates a sectional view of a catheter assembly of a device having heater circuit without a semi-porous thermal chamber, in accordance with some embodiments of the present specification;

FIG. 2D illustrates a system having the catheter assembly with a semi-porous thermal chamber shown in FIG. 2A;

FIG. 2E illustrates an exemplary design of an electrode configuration, used for heating an ablation fluid for hydrothermal ablation, in accordance with some embodiments of the present specification;

FIG. 2F separately illustrates the different layers used to form the electrode configuration of FIG. 2E, in accordance with some embodiments of the present specification;

FIG. 3A illustrates a distal portion of the catheter FIG. 1 with outer balloon, perforated layer and ablation balloon, in an expanded configuration;

FIG. 3B illustrates the distal portion of catheter of the FIG. 1 with outer balloon, perforated layer and ablation balloon, in a compressed configuration;

FIG. 4A illustrates at least four different embodiments of the sheet used to construct a perforated layer between inner and outer balloons of an ablation catheter;

FIG. 4B illustrates corresponding ablation patterns formed as a result of selecting each one of embodiments shown in FIG. 4A;

FIG. 5A is a flowchart illustrating the steps involved in one embodiment of a method of using the ablation system of FIG. 1 to ablate a body tissue;

FIG. 5B is a flowchart illustrating the steps involved in one embodiment of a method of using the ablation system of FIG. 2C to ablate a body tissue;

FIG. 6A illustrates a known structure of an artery;

FIG. 6B illustrates a known structure of a vein;

FIG. 7 illustrates position of distal portion of an ablation catheter within a renal artery, in accordance with some embodiments of the present specification;

FIG. 8 is a flow chart illustrating an exemplary process for renal denervation in accordance with some embodiments of the present specification;

FIG. 9A illustrates a distal end of an ablation catheter positioned within a renal artery, in accordance with some embodiments of the present specification;

FIG. 9B illustrates the distal end of the catheter of FIG. 9A with an expanded configuration of an outer balloon;

FIG. 9C illustrates the distal end of the catheter of FIG. 9B with an expanded configuration of a perforated layer;

FIG. 9D illustrates the distal end of the catheter of FIG. 9C with an expanded configuration of an inner ablation balloon;

FIG. 9E shows areas of therapeutic and sub-therapeutic ablation in a pattern that is based on the pattern of perforated layer of the ablation catheter of FIG. 9A, after ablation is performed;

FIG. 10 illustrates an example of geometry of ablation using the embodiments of the present specification;

FIG. 11A illustrates an exemplary spiral ablation geometry achieved in accordance with some embodiments of the present specification;

FIG. 11B illustrates an exemplary linear ablation geometry achieved in accordance with some embodiments of the present specification;

FIG. 11C illustrates a cross-section view of a linear ablation geometry showing the ablation area and depth or volume to be greater towards the cephalad portion, relatively less in the region between the cephalad and caudad, and very little or no ablation towards the caudad portion;

FIG. 11D illustrates a sample anatomy of renal sympathetic fibers spread over an aorta and extending across cephalad region of renal arteries;

FIG. 12A illustrates the position of a catheter of an ablation system in a renal artery;

FIG. 12B illustrates results of a spiral ablation using the ablation system of FIG. 12A;

FIG. 13A illustrates an exemplary embodiment of dimensions of an outer balloon that is used at the distal end of the catheter for ablation of renal artery, vein and/or nerves, in accordance with the present specification;

FIG. 13B illustrates an exemplary embodiment of dimensions of an inner ablation balloon that is used inside outer balloon of FIG. 13A, in accordance with the present specification;

FIG. 13C illustrates an exemplary configuration of outer balloon that can be achieved with inner ablation balloon, in their expanded states and during an ablation process, in accordance with some embodiments of the present specification;

FIG. 13D illustrates a non-continuous pattern created by positioning a selectively patterned inner balloon within an outer balloon, in accordance with some embodiments of the present specification;

FIG. 14 illustrates a side perspective view of an inner balloon configuration in a shape of a linear cylindrical tube, in accordance with an embodiment of the present specification;

FIG. 15 illustrates a front view of inner balloon of FIG. 14;

FIG. 16 illustrates a front view of another exemplary configuration of an inner balloon, in accordance with an embodiment of the present specification;

FIG. 17A illustrates a front view of another embodiment of an inner balloon configuration, in accordance with an embodiment of the present specification;

FIG. 17B illustrates a side perspective view of inner balloon of FIG. 17A;

FIG. 17C illustrates a side view of balloon of FIG. 17A;

FIG. 17D illustrates a side view of a catheter having a single balloon, wherein the balloon is not enclosed within an outer balloon, in accordance with an embodiment of the present specification;

FIG. 17E illustrates a side perspective view of the balloon shown in FIG. 17D;

FIG. 17F illustrates a cross sectional view of the balloon shown in FIG. 17D;

FIG. 17G illustrates an ablation catheter have a spiral shaped inner balloon within an outer balloon, in accordance with some embodiments of the present specification;

FIG. 18A illustrates an exemplary outer balloon in its expanded state, in accordance with some embodiments of the present specification;

FIG. 18B illustrates an inner balloon with an irregular or spiral shape in the expanded state, in according with some embodiments of the present specification;

FIG. 18C illustrates a front view of an inner ablation balloon in its expanded state for ablation with outer balloon of FIG. 18A;

FIG. 18D illustrates a side view of a first embodiment of inner ablation balloon;

FIG. 18E illustrates a side view of a second embodiment of inner ablation balloon;

FIG. 18F illustrates an exemplary spiral ablation geometry achieved using the balloon configurations of FIGS. 13A-13C and 18B, in accordance with some embodiments of the present specification; and

FIG. 18G illustrates an exemplary linear ablation geometry achieved using the balloon configurations shown in FIGS. 14A-17C, 18A, and 18C-18E, in accordance with some embodiments of the present specification.

### DETAILED DESCRIPTION

The devices and methods of the present specification can be used to cause controlled focal or circumferential ablation of targeted tissue to varying depth in a manner in which desired ablation is followed by complete healing without the occurrence of thrombosis, stenosis, char or blood coagulum formation. The dose and manner of treatment can be adjusted based on the type of tissue and the depth of ablation needed. The ablation device can be used for the treatment of hypertension, diabetes, nonalcoholic steatohepatitis/nonalcoholic fatty liver disease (NASH/NAFLD), asthma, and for the treatment of any mucosal, submucosal or circumferential lesion, such as inflammatory lesions, tumors, polyps and vascular lesions. The ablation device can also be used for the treatment of focal or circumferential mucosal, submucosal, or adventitial lesions or nerves supplying these structures of any hollow organ or hollow body passage in the body. The hollow organ can be one of a vascular structure such as blood vessels, heart tissue, pulmonary artery or vein, pulmonary vein ostium, left atrium, ventricular tissue, left atrial appendage, renal artery/vein/nerve, hepatic artery/vein/nerve or portal vein and bronchus or bronchial nerve, hepatic or celiac, artery, vein or nerve. The ablation device can be placed endoscopically, radiologically, surgically or under direct visualization. In various embodiments, a wireless camera or single fiber camera can be incorporated as a part of the device. In another embodiment, magnetic or stereotactic navigation can be used to navigate the catheter to the desired location. Radio-opaque or sonolucent material can be incorporated into the body of the catheter for radiological localization. Ferro- or ferromagnetic materials can be incorporated into the catheter to help with magnetic navigation.

The flow of the ablative agent is controlled by a microprocessor according to a predetermined method based on the characteristic of the tissue to be ablated, required depth of ablation, and distance of the device end from the tissue. The microprocessor may use temperature, pressure, electrical, impedance or other sensing data to control the flow of the ablative agent. The targeted segment can be treated by continuous ablation or via cycles of ablation as determined and controlled by the microprocessor.

It should be appreciated that the devices and embodiments described herein are implemented in concert with a controller that comprises a microprocessor executing control instructions. The controller can be in the form of any computing device, including desktop, laptop, and mobile device, and can communicate control signals to the ablation devices in wired or wireless form.

The present invention is directed towards multiple embodiments. The following disclosure is provided in order to enable a person having ordinary skill in the art to practice the invention. Language used in this specification should not be interpreted as a general disavowal of any one specific embodiment or used to limit the claims beyond the meaning of the terms used therein. The general principles defined herein may be applied to other embodiments and applications without departing from the spirit and scope of the invention. Also, the terminology and phraseology used is for the purpose of describing exemplary embodiments and should not be considered limiting. Thus, the present invention is to be accorded the widest scope encompassing numerous alternatives, modifications and equivalents consistent with the principles and features disclosed. For purpose of clarity, details relating to technical material that is known in the technical fields related to the invention have not been described in detail so as not to unnecessarily obscure the present invention.

“Treat,” “treatment,” and variations thereof refer to any reduction in the extent, frequency, or severity of one or more symptoms or signs associated with a condition.

“Duration” and variations thereof refer to the time course of a prescribed treatment, from initiation to conclusion, whether the treatment is concluded because the condition is resolved or the treatment is suspended for any reason. Over the duration of treatment, a plurality of treatment periods may be prescribed during which one or more prescribed stimuli are administered to the subject.

“Period” refers to the time over which a “dose” of stimulation is administered to a subject as part of the prescribed treatment plan.

The term “and/or” means one or all of the listed elements or a combination of any two or more of the listed elements.

In the description and claims of the application, each of the words “comprise” “include” and “have”, and forms thereof, are not necessarily limited to members in a list with which the words may be associated. The terms “comprises” and variations thereof do not have a limiting meaning where these terms appear in the description and claims. It should be noted herein that any feature or component described in association with a specific embodiment may be used and implemented with any other embodiment unless clearly indicated otherwise.

Unless otherwise specified, “a,” “an,” “the,” “one or more,” and “at least one” are used interchangeably and mean one or more than one.

The term “controller” refers to an integrated hardware and software system defined by a plurality of processing elements, such as integrated circuits, application specific integrated circuits, and/or field programmable gate arrays, in data communication with memory elements, such as random access memory or read only memory where one or more processing elements are configured to execute programmatic instructions stored in one or more memory elements.

The term “vapor generation system” refers to any or all of the electrode or heater approaches to generating steam from water described in this application.

The term “ablative agent” refers to any one or all of thermal fluid, cryogen, hot water, steam, or vapor, any of which can be mixed with a contrast agent. The concentration of the contrast may be 5% to 100%.

The term “cooling agent” refers to any one or all of CO₂, air, water, or saline, any of which may be mixed with a contrast agent. The concentration of the contrast may be 5% to 100%.

The terms “coolant”, “cooling agent”, and “insulating agent” may be used interchangeably and may refer to air, water, radiopharmaceutical contrast agent, or CO₂.

The term “ablative zone” refers to regions on the outer balloon or target tissue where temperature exceeds a predetermined therapeutic threshold (for example, ≥55° C. for ≥2 s for heating, or ≤−20° C. for ≥2 s for cryogenic treatment), measured under specified inflation and medium conditions.

The terms “non-ablative zone” or “sub-therapeutic zone” refer to a region not meeting the ablative criteria under the same conditions listed above.

For any method disclosed herein that includes discrete steps, the steps may be conducted in any feasible order. And, as appropriate, any combination of two or more steps may be conducted simultaneously.

Also herein, the recitations of numerical ranges by endpoints include all numbers subsumed within that range (e.g., 1 to 5 includes 1, 1.5, 2, 2.75, 3, 3.80, 4, 5, etc.). Unless otherwise indicated, all numbers expressing quantities of components, molecular weights, and so forth used in the specification and claims are to be understood as being modified in all instances by the term “about.” Accordingly, unless otherwise indicated to the contrary, the numerical parameters set forth in the specification and claims are approximations that may vary depending upon the desired properties sought to be obtained by the present specification. At the very least, and not as an attempt to limit the doctrine of equivalents to the scope of the claims, each numerical parameter should at least be construed in light of the number of reported significant digits and by applying ordinary rounding techniques.

Notwithstanding that the numerical ranges and parameters setting forth the broad scope of the specification are approximations, the numerical values set forth in the specific examples are reported as precisely as possible. All numerical values, however, inherently contain a range necessarily resulting from the standard deviation found in their respective testing measurements.

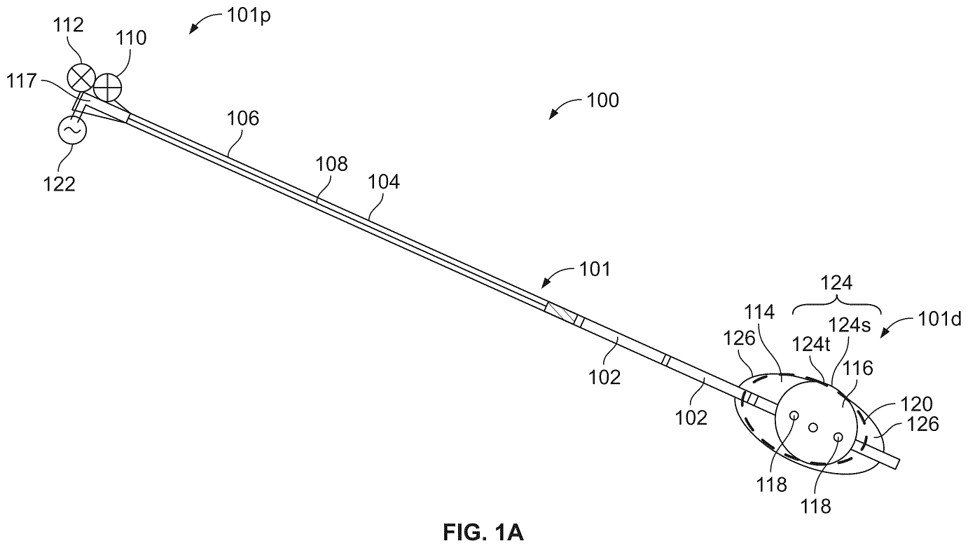

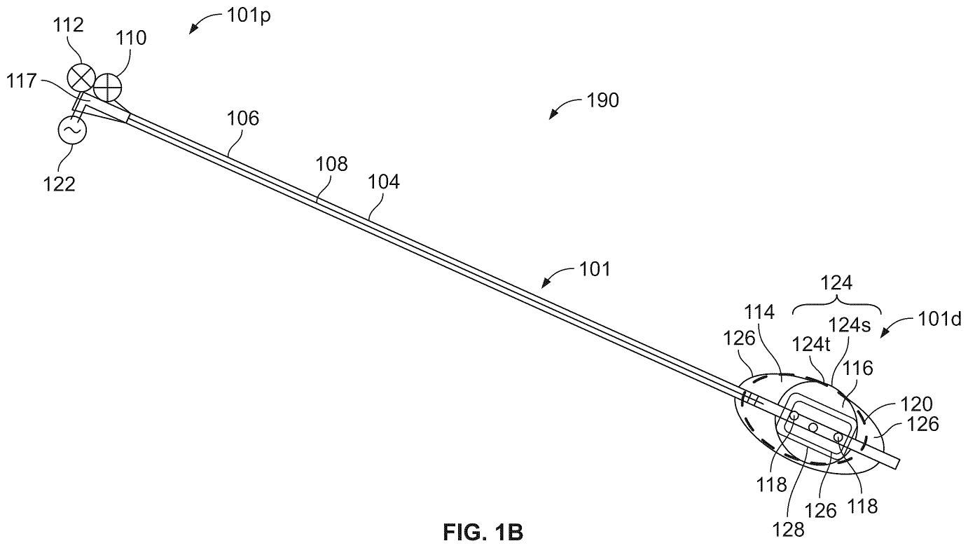

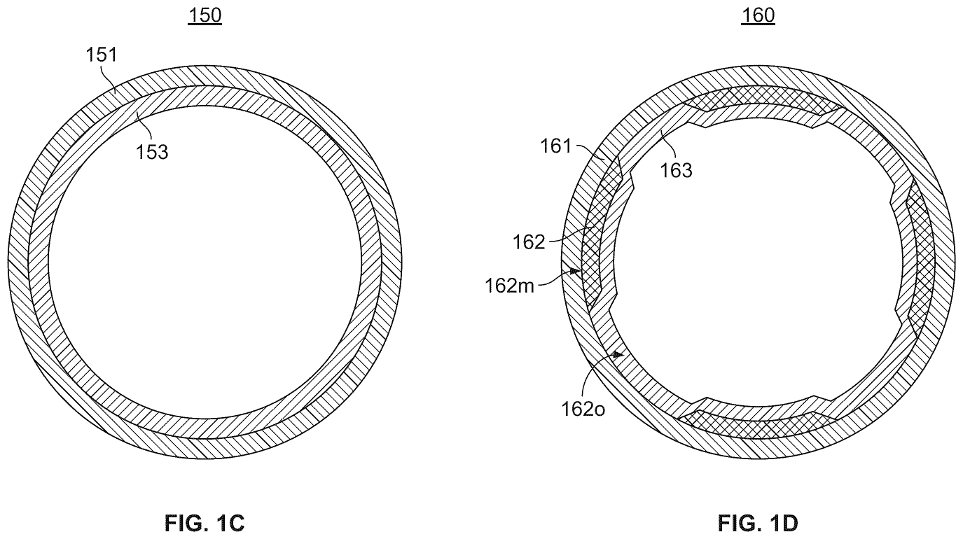

FIG. 1A illustrates an ablation system 100 having a catheter 101 with an inner balloon 116 and an outer balloon 114 and an intermediate or intervening layer 120 between the two balloons, in accordance with an embodiment of the present specification. FIG. 1B illustrates an ablation system 190 having a catheter 101 with an inner balloon 116 and an outer balloon 114 and an intervening layer 120 between the two balloons, and a semi-porous thermal chamber 128 within the inner balloon, in accordance with some embodiments of the present specification. The ablation system of FIG. 1A includes at least one heating chamber 102 positioned within the second lumen 108 while the ablation system of FIG. 1B include a semi-porous thermal chamber 128 within the inner balloon 116 and does not include any heating chambers within the catheter body/lumens. Referring to both FIGS. 1A and 1B, catheter 101 includes an elongate catheter body 104 and has a proximal end 101p and a distal end 101d with a first lumen 106 and a second lumen 108 within. In embodiments, the first lumen 106 is an air lumen and is in in fluid communication with a first fluid pump 110 at the proximal end 101p of the catheter 101. In some embodiments, the first fluid pump 110 is an air pump. In embodiments, the second lumen 108 is a water/saline/vapor lumen and is in fluid communication with a second fluid pump 112 at the proximal end 101p of the catheter 101. In some embodiments, the second fluid pump is a water or saline pump. The first lumen 106 is in fluid communication with an outer balloon 114 attached to the distal end 101d of the catheter 101.

In embodiments, the intervening layer 120 may be a mesh, stencil, mask, cutout, overlay, matrix, lattice, grid, linear strips, spiral strips, ribs, dots, pads, gradients, or any material layer, made of a metallic wire, for example, Nitinol, stainless, steel, or copper, or a polymeric plastic or thermoplastic, for example polyetheretherketone (PEEK), polytetrafluoroethylene (PTFE), polyethylene terephthalate (PET), Pebax, parylene, or nylon, polymer-coated metal, or any combination thereof, positioned between the outer surface of the inner balloon 116 and the inner surface of the outer balloon 114 and adapted to create a pattern having an area that is less than the area of an annular ring that would have been formed by the inner balloon directly contacting the outer balloon in the absence of the intermediate layer but having a periphery that is more than the periphery of said annular ring. In embodiments, the intervening layer has a plurality of open and closed areas created by the pattern of the layer. The open areas are defined as opening or reduced thickness portion through which the inner balloon can directly or near directly contact the outer balloon under inflation. The closed areas are defined as material-covered portion that maintains a standoff or thermal barrier between balloons. In various embodiments, a ratio of open areas to closed areas is in a range of 10% to 90% open areas to closed areas. In some embodiments, a ratio of open to close areas, or open area fraction (OAF) is ≤50%, yielding an ablative area of ≤50% of the contact of the outer balloon with tissue. In other embodiments where OAF>50%, the structure mechanically restricts local expansion and/or contact so that the ablative area is <50% of the non-ablative area under defined inflation conditions. In embodiments, “restricts expansion” is defined as the intermediate layer reduces local radial displacement and/or contact pressure sufficient to keep the region below the ablative criterion under said inflation conditions. In some embodiments, OAF is computed from plan view images of the nominal annular band at a defined inner balloon pressure Pᵢₙ, outer balloon pressure Pₒᵤₜ, and medium temperature Tₘ, using binary segmentation of open versus material covered regions. In some embodiments, the pattern generated by the intermediate layer has an area less than the annular area that would form absent the intermediate layer and a periphery greater than that annulus, increasing edge length and discretization without increasing total area. In some embodiments, an area of ablative zones created by using the intermediate layer is <40%, <33%, or <25% of an area of non-ablative zones. In various embodiments, the intermediate or intervening layer is manufactured by laser cutting or die cutting of films, braiding or weaving, additive printing, photolithographic parylene deposition, over molding or adhesive bonding of strips or pads, placement on the inner balloon, the outer balloon, or free floating between them. Due to the patterning and when the inner balloon 116 is inflated with ablative fluid and contacts the outer balloon 114, the intermediate or intervening layer 120 forms at least two zones in the area of contact between the inner balloon and the outer balloon, creating an ablative zone where there is no intermediate layer present and a non-ablative zone where the intermediate layer is present. In embodiments, a temperature differential between the ablative zone and the non-ablative zone is at least 5° C., 10° C., or 20° C. In embodiments, the temperature differential is measured at the outer balloon surface 10 seconds after inflation under specified inflation pressures. In embodiments, the intervening layer 120 is entirely made from a metallic wire, for example, Nitinol, stainless, steel, or copper, or a plastic or thermoplastic, for example PEEK, PTFE, or Nylon, or any combination thereof, and, in one embodiment, does not contain, comprise, or contact any electrodes or other energy delivery sources. In various embodiments, the intervening layer has a thickness that varies from 50 μm to 250 μm.

When an inner balloon is positioned concentrically within an outer balloon and inflated with a heated medium, the inflation produces a circumferential contact band. In the absence of modification, this contact assumes the form of a continuous annulus. FIG. 1C illustrates a continuous annulus 150 formed from an inflated inner balloon 153 positioned within an inflated outer balloon 151 with no intermediate layer or intervening layer positioned between. The annulus 150 has a radial thickness or width defined between the outer surface of the inner balloon 153 and the inner surface of the outer balloon 151 at the circumferential line of tangency. Due to balloon compliance, material elasticity, and inflation pressure, the annular width varies no more than +10% along the circumference, thus representing a substantially uniform circular or annular contact region.

To convert this continuous annular contact into a non-continuous pattern 160, referring to FIG. 1D, a selectively patterned intervening or intermediate layer 162 is disposed between the inner balloon 163 and the outer balloon 161. The intermediate layer includes portions of material 162m intermixed with openings 1620 (or reduced-thickness regions). At material-covered portions, the layer prevents direct contact between the inner and outer balloons, thereby reducing or eliminating conductive energy transfer at those positions and resulting in sub-therapeutic or non-ablative energy delivery. At openings, gaps, or places of reduced thickness relative to the material-covered portions, the inner balloon directly or very closely contacts the outer balloon, concentrating thermal transfer to form localized therapeutic or ablative energy delivery. The resulting interface no longer produces a continuous annulus but instead generates a discretized contact pattern comprising ablative regions separated by, or broken up by, non-ablative regions.

The discrete ablative regions may be regularly or irregularly spaced along the circumference. In one example, the openings form a repeating pattern of radial segments, thereby creating a “dashed” annular contact. In another embodiment, the openings form a two-dimensional array, such as a checkerboard, spiral, starburst, polygon, or lattice pattern, producing a pixelated ring of ablative sites. The discretization defines a therapeutic map in which: each ablative region corresponds to a direct-contact, or almost direct contact, zone having sufficient thermal transfer for ablation; and each intervening region corresponds to an insulated zone where direct balloon-to-balloon contact is sufficiently blocked, thereby providing sub-therapeutic energy exposure.

This conversion from continuous to discretized contact enables a) controlled lesion patterning that produces multiple ablation sites of varying width and/or multiple non-contiguous ablation sites rather than a uniform circumferential lesion, b) dose modulation reducing overall thermal load on surrounding tissues by limiting the extent of direct ablation, c) spatial customization tailoring the lesion geometry through choice of pattern (e.g., radial slits, perforations, lattices), and d) enhanced safety by decreasing risk of over-treatment or circumferential scarring by creating controlled spacing between lesions.

In some embodiments, the intermediate layer functions as a passive thermal mask and comprises a thin, flexible aerogel-based film that exhibits high thermal resistance at minimal thickness while remaining conformable during balloon folding, track, and inflation. In one embodiment, the intermediate layer is formed from a polyimide aerogel film commercially available as AeroZero®. A single film having a nominal thickness of about 100-250 μm, more preferably about 165 μm, is patterned to include open regions and closed (material-covered) regions. The film exhibits low thermal conductivity at room temperature and maintains mechanical integrity and dielectric properties at temperatures associated with heated-fluid or vapor ablation. When applied as a laminate, the film may be supplied with a pressure-sensitive adhesive (PSA), such as a silicone PSA, having a thickness of about 10-35 μm. To minimize parasitic heat flow, the adhesive is disposed only beneath the closed regions of the pattern or as peripheral bonding rails, leaving the open regions substantially free of adhesive. In this configuration the laminate preserves the high areal thermal resistance of the aerogel layer while providing robust bonding to the balloon substrate and reliable laser-cut edge quality for fine features.

In some embodiments, the intermediate layer is patterned as a stencil or mask so that the plan-view open-area fraction within the nominal annular contact band is between about 10% and 90%. Sub-ranges may be specified to tailor lesion density, including less than 25%, 25-50%, 50-75%, and greater than 75%. Open regions are arranged to permit near-direct or direct contact between the balloons, thereby concentrating conductive energy transfer and defining ablative zones. Closed regions interpose the aerogel material between the balloons, attenuate the conductive path, and define sub-therapeutic zones. Under standard inflation conditions, the arrangement produces a measurable temperature differential between ablative and sub-therapeutic zones; in certain embodiments the minimum differential is at least 5° C., 10° C., or 20° C. at a fixed time after inflation as specified elsewhere in this description.

For applications requiring higher thermal contrast at minimal added profile, multiple aerogel films may be stacked to form a composite (e.g., a dual-film or triple-film laminate), separated by one or more thin PSA interfaces. Such stacks increase the areal thermal resistance approximately in proportion to the total aerogel thickness while maintaining an overall thickness below about 0.55 mm, thereby preserving catheter deliverability. The patterned geometry of the intermediate structure may take the form of linear strips, spiral strips of one to four starts, meshes or lattices (periodic or aperiodic), discrete ribs or pads forming a dashed annulus, or combinations thereof. Gradients in open-area fraction or local thickness may be incorporated around the circumference to further control lesion spacing.

Alternative materials may be substituted for the AeroZero® film while maintaining the functional characteristics of flexibility, thin profile, and high thermal resistance. Suitable commercial alternatives include silica-aerogel composite sheets such as Panasonic NASBIS® provided in thicknesses on the order of 50-1000 μm; polyimide-aerogel films such as Airloy® X-series flexible films; and multilayer laminates of aerogel films with polymeric cover layers for abrasion resistance. Research-grade polymer aerogels, including polyimide aerogel films and aramid-nanofiber aerogel films fabricated via sol-gel, phase-separation, or freeze-casting methods, may also be used where manufacturing scale and biocompatibility allow. Any of the foregoing may be employed singly or in stacks, optionally with polymeric cover layers (e.g., polyimide) or barrier coatings (e.g., parylene) to improve handling, tear resistance, or solvent resistance, provided that the total thickness remains compatible with catheter folding and track forces.

The intermediate structure is passive and is not configured to deliver energy. In at least one embodiment, the intermediate structure does not contain, comprise, or contact an electrode, resistive heater, microwave radiator, radio-frequency conductor, or any other energy-delivery source; the patterned energy application arises from thermo-mechanical masking effected by the material's thermal resistance. The structure may be bonded to the outer surface of the inner balloon, to the inner surface of the outer balloon, or may be retained as a free-floating element captured between the balloons, with the adhesive distribution selected to preserve the designed open-area fraction and to avoid creating unintended thermal bridges.

Manufacturing may include laser micromachining or die cutting of the aerogel film prior to lamination. Laser parameters are selected to limit the heat-affected zone to within about 50 μm of a feature edge. Lamination is performed at temperatures and pressures compatible with the aerogel's porous structure so as not to collapse the pores or significantly increase thermal conductivity. Where required for assembly robustness, a thin polymer cover film (e.g., 12-25 μm polyimide) may be co-laminated over the aerogel on the non-contact side to enhance handling without materially degrading the areal thermal resistance.

For purposes of measurement within this specification, open-area fraction is computed from plan-view imaging of the nominal annular band at defined inner-balloon and outer-balloon pressures and at a defined medium temperature. A region is deemed “ablative” if its surface temperature on the outer balloon meets or exceeds a specified therapeutic threshold for a specified dwell time; all measurements are taken at a fixed time point after inflation to ensure reproducibility. Areal thermal resistance of the intermediate structure is calculated as the sum of layer thickness divided by layer thermal conductivity for each constituent of the laminate, with adhesive contributions minimized by the placement strategy described above.

The outer balloon 114 may include a plurality of optional sensors within or attached to the outer surface of its walls. In embodiments, the sensors are configured to determine either temperature, pressure or electrical impedance for measuring tissue signals and aiding in monitoring/controlling the ablation process. In some embodiments, the outer balloon 114 includes up to 24 sensors.

The second lumen 108 is in fluid communication with an inner balloon 116 attached to the distal end 101d of the catheter 101 and positioned within the outer balloon 114. In the present specification, the terms inner balloon and ablation balloon are used interchangeably. A plurality of infusion ports 118 are included in a portion of the distal end of the catheter body wherein the ablation balloon 116 is attached to allow for the passage of heated vapor from the second lumen 108 into the inner or ablation balloon 116. The system 100 includes a controller 117 configured to cause the first fluid pump 110 to deliver fluid, via the first lumen 106, to the outer balloon 114 to change the outer balloon from a first state, in a compressed, deflated configuration, to a second state, in an expanded, inflated configuration. The controller is also configured to cause the second fluid pump 112 to deliver water, saline, or a diluted contrast solution to the second lumen 108, where at least one heating chamber 102 (referring to FIG. 1A) positioned within the second lumen 108 is configured to provide thermal energy to the water, saline or contrast solution to convert the water, saline or contrast solution to heated fluid, which is then delivered via ports 118 to the ablation balloon 116. In embodiments, the at least one heating chamber 102 (referring to FIG. 1A) comprises at least one or an array of electrodes configured to receive an RF current from RF generator 122 to heat the electrode and convert the water, saline or contrast solution to hot fluid or hot water vapor. Heated fluid or water vapor provided to the ablation balloon causes the ablation balloon to change from a first state, in a compressed deflated configuration, to a second state, in an expanded, inflated configuration. Areas where the inflated ablation balloon 116 contacts the inflated outer balloon 114 comprise hot or ablation zones 124 and provide for the transfer of ablative energy from the ablation balloon 116 to the target tissue for ablation. In another embodiment, a plurality of infusion ports 118 are included in a portion of the distal end of the catheter body wherein the ablation balloon 116 is attached to allow for the passage of a cold ablative agent from the second lumen 108 into the inner or ablation balloon 116. The cold ablative agent provided to the ablation balloon 116 causes the ablation balloon 116 to change from a first state, in a compressed deflated configuration, to a second state, in an expanded, inflated configuration. Areas where the inflated ablation balloon 116 contacts the inflated outer balloon 114 comprise cold or ablation zones 124 and provide for the transfer of ablative energy from the ablation balloon 116 to the target tissue for ablation.

In embodiments, an intervening layer 120 is attached to the distal end 101d of the catheter 101 and positioned between the ablation balloon 116 and outer balloon 114. In embodiments, the intervening layer is perforated. In the present specification, intervening layer and perforated layer are used interchangeably. Perforated layer 120 is an expandable perforated structure that contracts and is configured to expand from a first state in a compressed configuration to a second state in an expanded configuration, similarly to outer balloon 114 and ablation balloon 116. As the name suggests, perforated layer 120 is constructed using a sheet comprising perforations (holes or gaps) provided in an otherwise contiguous sheet. The contiguous portion of perforated layer 120 blocks ablative energy from ablation balloon 116 to pass to outer balloon 114, whereas the portion of perforated layer 120 comprising perforations allows the ablative energy to escape and reach outer balloon 114 and thereafter the target tissue contacting or proximate the outer balloon 114. The regions where the ablative energy reaches outer balloon 114 from ablation balloon 116 creates areas of therapeutic ablation. The regions where the ablative energy is blocked from reaching outer balloon 114 creates areas of subtherapeutic ablation. In an embodiment, a temperature in the areas of therapeutic ablation is above 50° C. and that in the areas of subtherapeutic ablation is less than 45° C., in cases where a hot ablation agent is used. In an embodiment, a temperature in the areas of therapeutic ablation is less than 10° C. and that in the areas of subtherapeutic ablation is greater than 25° C., in cases where a cold ablation agent is used. The non-contiguous ablation pattern created with the addition of the intervening perforated layer 120 prevents extensive scarring and stricture formation in a human tissue.

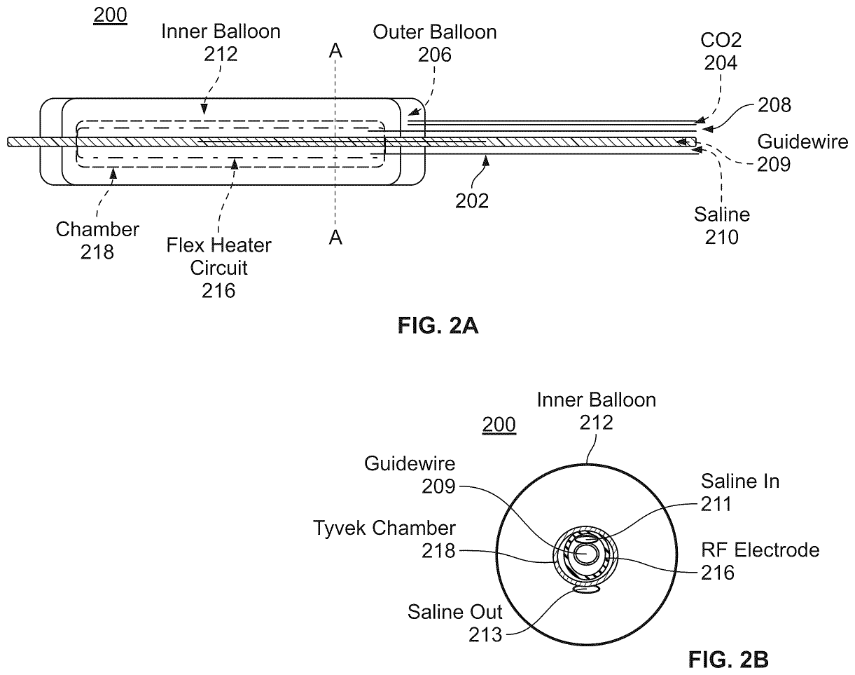

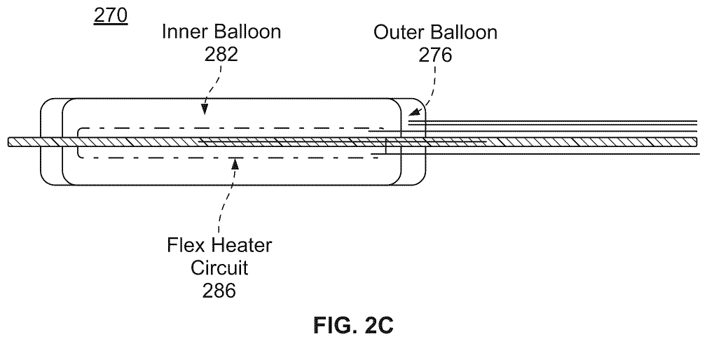

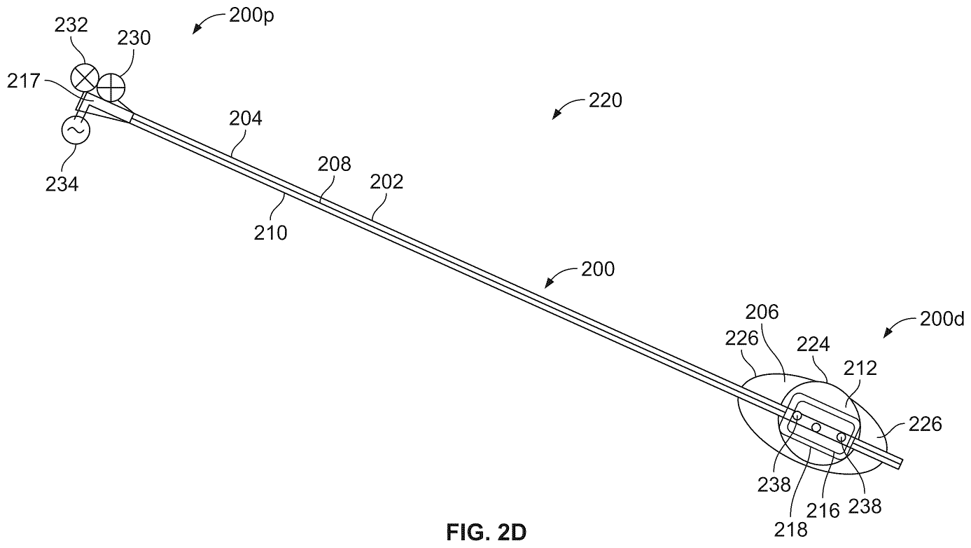

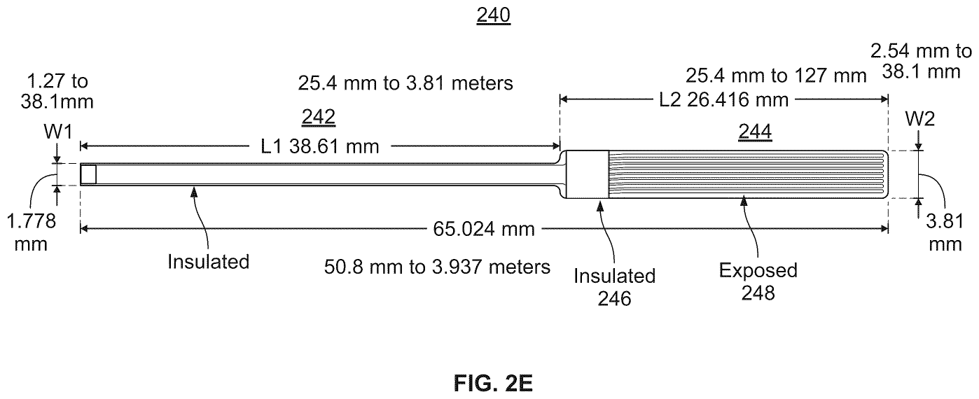

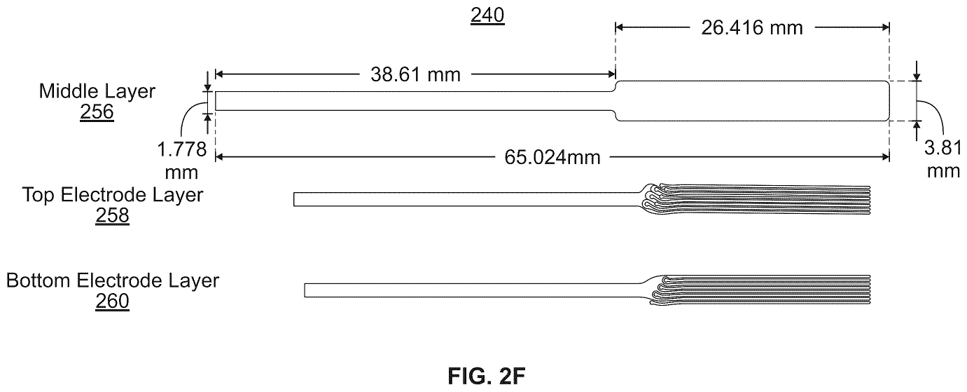

FIG. 1B is an embodiment similar to that described with reference to FIG. 1A, however, the catheter 101 does not include heating chambers 102 (described with reference to FIG. 1A) in the catheter body but instead includes a semi-porous chamber 128 within the inner balloon 116. The semi-porous chamber 128 is similar to that described with reference to FIGS. 2A-2D and includes a heater circuit 126 within. In embodiments, the heater circuit includes electrodes similar to those described with reference to FIGS. 2E-2F.

### Thermal Chambers

In embodiments, a plurality of electrodes, configured as one or more arrays of electrodes is disposed between an outer covering and an inner coaxial core, to form a thermal or heating chamber. For an optimal positioning of the electrode, a primary requirement is that the electrode is positioned proximal to the output of the hot fluid channel into the inner balloon. At the same time, it is desirable that the electrode is not too proximal, because the quality of steam quality (the amount of condensed water in the steam) decreases. In embodiments, the temperature of superheated steam is ˜100° C.+20° C. and the steam quality ranges from 100% dry steam to >25% dry steam. In other embodiments, the ablative agent is a hot liquid or hot steam. In alternate embodiments, the thermal chambers of the present specification do not include electrodes and are configured to receive a cryo-agent for delivering thermal ablation to a target tissue.

In embodiments, the outer covering and the inner core are comprised of silicone, Teflon, ceramic or any other suitable thermoplastic elastomer known to those of ordinary skill in the art. The inner core, outer covering, and electrodes are all flexible to allow for bending of the distal portion or tip of the catheter to provide better positioning of the catheter during ablation procedures. In embodiments, the inner core stabilizes the electrodes and maintains the separation or spacing between the electrodes while the tip of the catheter flexes or bends during use.

In some embodiments, electrode is positioned within the inner balloon. In some embodiments, the electrode is positioned a distance in a range of 0 mm to 500 mm proximal to the output of the hot fluid channel into the inner balloon. In some embodiments, the electrode is positioned at a distance in a range of 1 mm to 150 mm proximal to the output of the hot fluid channel into the inner balloon.

In accordance with an aspect of the present specification, multiple heating chambers can be arranged in the catheter tip. In some embodiments, two heating chambers are positioned in a series. In some embodiments, the electrode is made of a plurality of segments which are electrically connected to each other and housed in a flexible section of a catheter body with a desired bend radius wherein the length of each segment of the electrode is less than four times the bend radius of the catheter, thereby providing sufficient length coverage.

During vapor generation, a signal can be sensed to determine if the fluid has fully developed into vapor before exiting the distal end of the heating chamber. In some embodiments, the signal is sensed by a controller. Sensing whether the vapor is fully developed can be particularly useful for many surgical applications, such as in the ablation of various tissues, where delivering high quality fully developed vapor results in more effective treatment. In some embodiments, the heating chamber includes at least one sensor. In various embodiments, said at least one sensor comprises an impedance, temperature, pressure or flow sensor. In one embodiment, the electrical impedance of the electrode arrays can be sensed. In other embodiments, the temperature of the fluid, temperature of the electrode arrays, fluid flow rate, pressure, or similar parameters can be sensed.

It should be appreciated that any ablation catheter or system of the present specification, used to ablate tissue in an organ, may be used with a controller, wherein the controller is configured to limit a pressure generated by ablation fluid, such as hot water, steam, vapor, or a cryogen within the organ to less than 5 atm or 100 psi.

FIG. 2A illustrates a sectional view of a catheter assembly of a device having a semi-porous thermal chamber, in accordance with some embodiments of the present specification. FIG. 2B illustrates a cross-sectional view along an axis A-A shown in FIG. 2A, of catheter assembly 200. Referring simultaneously, to FIGS. 2A and 2B, a catheter shaft 202 includes multiple parallel lumens. A first lumen 204 provides a channel to deliver a fluid including saline/air/carbon dioxide (CO₂) to provide saline/air/CO₂ for inflating an outer balloon 206 that is attached to a distal end of shaft 202. The first lumen 204 is in fluid communication with the outer balloon 206. In embodiments, the first lumen 204 extends from a proximal end of the catheter assembly 200 to a first point before a distal end of the catheter assembly 200. A second lumen 208 passes centrally through shaft 202 to provide passage to a guidewire 209. Lumen 208 passes centrally through shaft 202 and through outer balloon 206 and inner balloon 212 to a distal side of balloons 206, 212. In some embodiments, the second lumen 208 extends from a proximal end of the catheter assembly 200 to a second point before or at a distal end of the catheter assembly 200. A third lumen 210 extends within shaft to deliver saline into a chamber 218 within an inner balloon 212. In embodiments, the chamber 218 is a semi-porous thermal chamber. The third lumen 210 is in fluid communication with the chamber 218 which, in turn, is in fluid communication with the inner balloon 212. In embodiments, the third lumen 210 extends from a proximal end of the catheter assembly 200 to a first point before a distal end of the catheter assembly 200. Referring to FIG. 2B, in some embodiments, the third lumen 210 is further divided into first subdivision lumen 211 for saline to enter the chamber 218 and second subdivision lumen 213 for saline to exit the chamber 218. In other embodiments, the saline enters and exits the chamber 218 through the same third lumen 210. A flex heater circuit 216 comprising at least one RF electrode is positioned within the chamber 218 that is positioned within the inner balloon 212. In some embodiments, chamber 218 is composed of Tyvek. In some embodiments, chamber 218 is composed of PTFE or GORTEX. In these embodiments, the layer of Tyvek, GORTEX or PTFE or a comparable material known in the art serves as a porous layer that allows for passage of the heated water vapor but doesn't allow for liquid or saline to pass into the inner balloon 212. The chamber 218 is in fluid communication with third lumen 210 and with the inner balloon 212 and is configured to receive saline through third lumen 210 and heat the saline to convert the saline to a vapor by heater circuit 216 and deliver the heated vapor to the inner balloon 212. Inner balloon 212 surrounds chamber 218, and is inflated by heated vapor to a diameter so that at least some portions of an outer surface of inner balloon 212 touches at least some portions of an inner surface of outer balloon 206 during an ablation process, creating an ablation zone to contact tissue.

In some embodiments, referring to FIG. 2C, a catheter 270 assembly does not include the chamber 218 as seen in FIG. 2A, and the flex circuit 286 is configured to directly heat the fluid in the inner balloon 282, wherein inner balloon 282 serves the function of the chamber 218 as seen in FIG. 2A. In this embodiment, hot water or hot saline may be the ablative agent. Also pictured in FIG. 2C is the outer balloon 276. The inner balloon is positioned within the outer balloon 276 and an ablation pattern is defined by the contact portions of the inner balloon 282 with the outer balloon 276 when both balloons are inflated.

FIG. 2D illustrates a system 220 having the catheter assembly shown in FIG. 2A. The system 220 comprises a catheter assembly 200 with an inner balloon 212 positioned within an outer balloon 206, in accordance with an embodiment of the present specification. A chamber 218 with a flex heater circuit 216 positioned within the chamber 218, is in turn positioned within the inner balloon 212. In certain embodiments, the inner balloon 212 serves as the chamber 218. Catheter 200 includes an elongate catheter body 202 and has a proximal end 200p and a distal end 200d with a first lumen 204, a second lumen 208, and a third lumen 210 within. In embodiments, the first lumen 204 is a fluid lumen to deliver saline/air/carbon dioxide (CO₂) to provide fluid for inflating the outer balloon 206, and is in in fluid communication with a fluid pump 230 at the proximal end 200p of the catheter 200. In embodiments, the second lumen 208 is a guidewire lumen for passage of a guidewire through the length of the catheter 200. In embodiments, the third lumen 210 is a fluid lumen for receiving saline from a saline pump 232 at the proximal end 200p of the catheter 200.

The second lumen 208 is in fluid communication with the chamber 218 within the inner balloon 212 attached to the distal end 200d of the catheter 200 and positioned within the outer balloon 206. In the present specification, the terms inner balloon and ablation balloon are used interchangeably. A plurality of infusion ports 228 are included in the chamber 218 and allow for the passage of heater vapor from the chamber 218 into the inner or ablation balloon 212. In certain embodiments the layer of Tyvek, GORTEX or PTFE serves as a porous layer that allows for passage of the heated water vapor but doesn't allow for liquid or saline to pass. The system 220 includes a controller 217 configured to cause the fluid/air/CO₂ pump 230 to deliver fluid, air or CO₂, via the first lumen 204, to the outer balloon 206 to change the outer balloon from a first state, in a compressed, deflated configuration, to a second state, in an expanded, inflated configuration. The controller is also configured to cause the saline pump 232 to deliver water or saline to the second lumen 208, where flex heater circuit 216 comprising at least one RF electrode positioned within the chamber 218 is configured to provide thermal energy to the water or saline to convert the water or saline to heated vapor or hot water, which is then delivered via ports 238 or through the porous layer of Tyvek to the inner ablation balloon 212. In embodiments where the inner balloon 212 functions as the chamber 218, the flex heater circuit 216 directly heats the water or saline in the balloon 212. In embodiments, the at least RF electrode of the flex heater circuit 216 is configured to receive an RF current from RF generator 234 to heat the electrode and convert the water or saline to hot water or heated vapor. Hot water or heated vapor provided to the ablation balloon causes the ablation balloon to change from a first state, in a compressed deflated configuration, to a second state, in an expanded, inflated configuration. Areas of the outer balloon 206 where the inflated ablation balloon 212 contacts the inflated outer balloon 206 comprise hot or ablation zones 224 and provide for the transfer of ablative energy from the ablation balloon 212 to the target tissue for ablation. Non-ablation zones 226, also referred to as cold zones or insulation zones, comprise areas of the outer balloon 206 where the inner balloon 212 and outer balloon 206 do not contact one another and have an average temperature that is different than an average temperature of ablation zones and is in the non-ablation range. In some embodiments, the system does not include chamber 218 and the heater circuit is configured to directly heat fluid within the inner balloon, as described with reference to FIG. 2C.

In certain embodiments, a cryoablation agent such as liquid CO₂ or liquid nitrogen is delivered to the inner balloon 212 and the areas of the outer balloon 206 where the cryo-inflated ablation balloon 212 contacts the inflated outer balloon 206 comprise cold or ablation zones 224 and provide for the transfer of ablative energy from the ablation balloon 212 to the target tissue for ablation. Non-ablation zones 226, also referred to as insulation zones, comprise areas of the outer balloon 206 where the inner balloon 212 and outer balloon 206 do not contact one another and have an average temperature that is higher than an average temperature of ablation zones and is in the non-ablation range.

FIG. 2E illustrates an exemplary design of an electrode configuration 240 used for heating an ablation fluid for thermal ablation in accordance with some embodiments of the present specification. The design comprises an elongated rectangular body with a first portion 242 having a first length L1 ranging from 25.4 mm to 3.81 meters, and is 38.61 mm in an embodiment, and having a first width W1 ranging from 1.27 to 38.1 mm, and is 1.778 mm in an embodiment. First portion is continually and linearly connected to a second portion 244 having a second length L2 ranging from 25.4 mm to 127 mm, and is 26.416 mm in an embodiment, and having a second width W2 ranging from 2.54 mm to 38.1 mm, which is 3.81 mm in an embodiment. Therefore, a total length of the electrode configuration ranges from 50.8 mm to 3.937 meters, and is 65.024 mm in an embodiment. Outer surface of first portion 242 is insulated, whereas a portion 246 of outer surface of second portion 244, which is proximal to first portion 242 is insulated, while the surface of the remaining portion 248 is exposed.

FIG. 2F separately illustrates the different layers used to form the electrode configuration 240 of FIG. 2D, in accordance with some embodiments of the present specification. Three layers form the configuration, comprising a middle layer 256 which is between a top electrode layer 258 and a bottom electrode layer 260. Base material of each electrode layer is 34.79 μm cu/25 μm polyimide/34.79 μm cu. A cover layer covering both sides of the electrode layers is 12 μm polyimide, which extends up to 25 μm of both sides. Parts of the electrode layers are copper plated. Finished thickness of each layer is approximately 34.79 μm cu.

The saline flow rate, passed within the heating chamber for conversion to vapor, may depend on size of the electrode, including the length, width, and periphery of the electrode and the power supplied. Generation of vapor or steam results in transfer of heat energy from the inner balloon through the outer balloon to ablate the target tissue. In some embodiments, contact of the inner and outer balloons at the ablation zone is documented and confirmed using fluoroscopy, 3D mapping and/or endoscopy. In some embodiments, one or more sensors in the ablation zone are utilized to monitor contact of the inner and outer balloons. In embodiments, the temperature of the inner balloon is monitored. During and after ablation, the temperature and pressure in the outer balloon is monitored to maintain at desired therapeutic values.

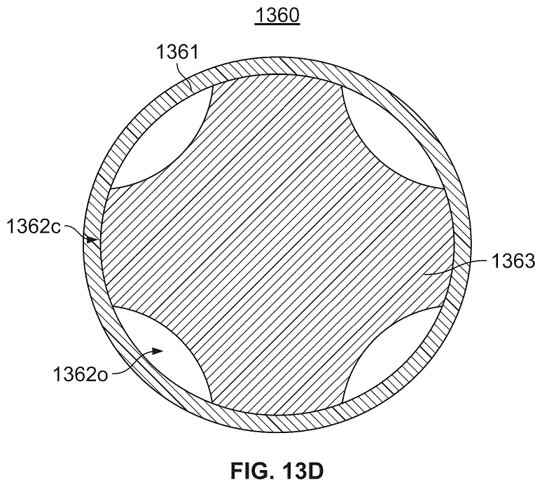

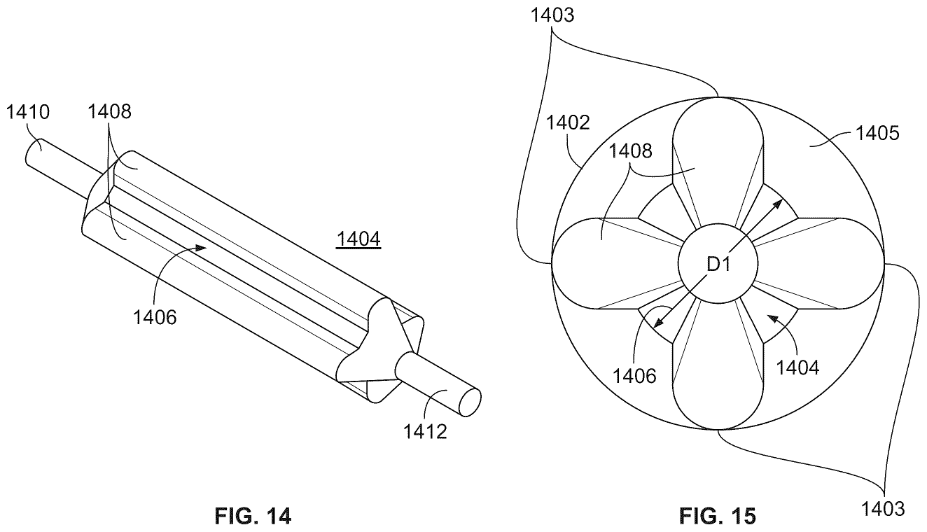

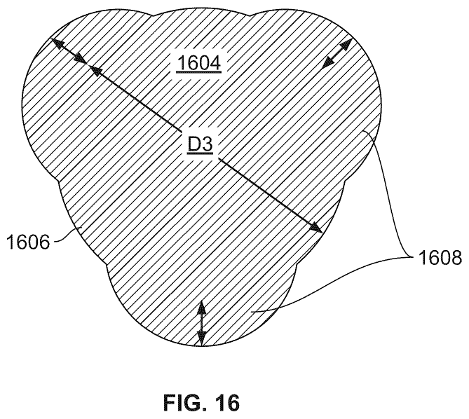

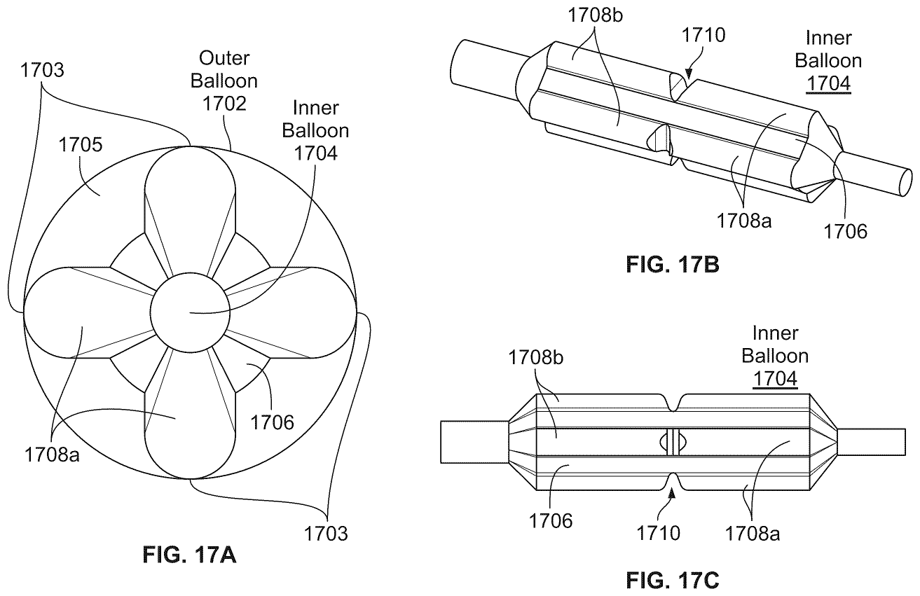

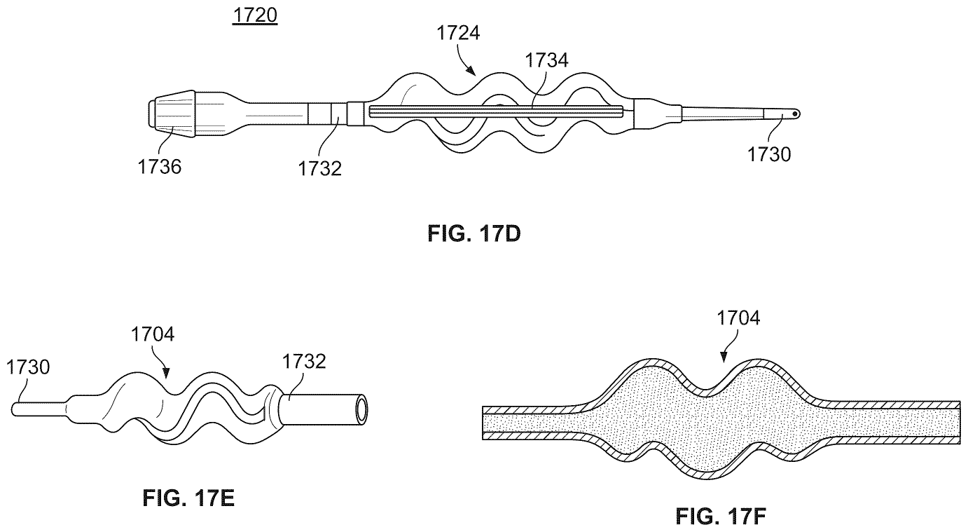

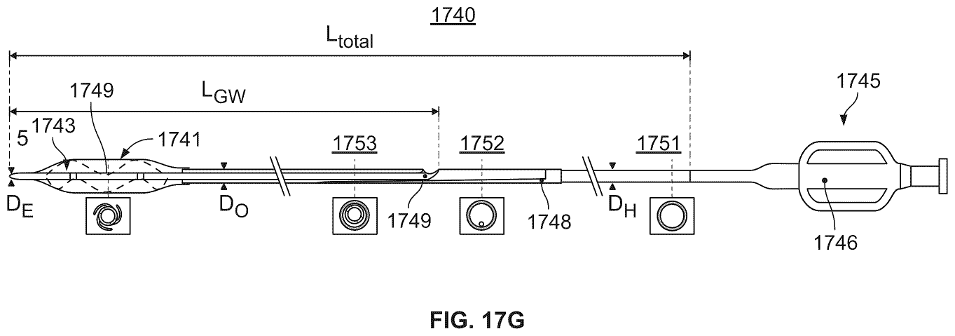

Referring to FIGS. 2A-2D, one or both of the inner balloon and outer balloon is configured in at least one of a circular, cylindrical, spiral, starburst or irregular shape. In some embodiments, for example, referring to FIGS. 14 and 15, the inner balloon has a cylindrical shape with linear extensions extending away from a central longitudinal axis of the inner balloon and the outer balloon has a cylindrical shape. In other embodiments, for example, referring to FIGS. 17D-17G, the inner balloon has a spiral shape and the outer balloon has a cylindrical shape or also has a spiral shape. Points of contact between the inner balloon and the outer balloon create non-circumferential areas for the transfer of ablative energy from the inner balloon to the outer balloon and to the target tissue.

### Ablation Catheters And Methods

In various embodiments, ablation catheters, having one or more inflatable balloons, are disclosed. In various embodiments, as detailed in Table 1 below, the one or more balloons are of the following types and specifications:

TABLE 1
Balloon Types and Specifications
Non-compliant Dilatation Balloons

| | |
|---|---|
| Characteristics | Ultrahigh strength, thin walls |
| Materials | PET |
| Compliance Range | 0%-10% (typical) |
| Sizes | Diameter: 0.5 mm-80 mm |
| | Length: Virtually any (15″ max) |
| Burst pressures | 15 psi-400 psi (1 atm-27 atm) |

Semi-compliant Dilatation Balloons

| | |
|---|---|
| Characteristics | High strength, thin walls |
| Materials | Nylon, Polyurethane, other thermoplastic elastomers |
| Compliance Range | 10%-20% (typical) |
| Sizes | Diameter: 0.5 mm-50 mm |
| | Length: 15″ max |
| Burst pressures | 15 psi-375 psi (1 atm-25.5 atm) |

Compliant Balloons

| | |
|---|---|
| Characteristics | Low pressure, thin and thick walls |
| Materials | Polyurethane, Nylon elastomers, and other |
| | thermoplastic elastomers |
| Compliance Range | 20%-200% or more |
| Sizes | Diameter: 0.5 mm-80 mm |
| | Length: Virtually any (15″ max) |
| Burst pressures | 0 psi-30 psi (0 atm-2 atm) |
| | Balloons can be designed for volume |

Following are some definitions with reference to balloon types and specifications:

Balloon Diameter—refers to nominal inflated balloon diameter measured at a specified pressure.

Balloon Length—typically refers to the working length or the length of the straight body section.

Burst Pressure—refers to an average pressure required to rupture a balloon; usually measured at body temperature.

Rated Burst Pressure—refers to a maximum statistically guaranteed pressure to which a balloon can be inflated without failing. For PTCA and PTA catheters, this is normally 95% confidence/99.9% guarantee.

Balloon Profile—refers to the maximum diameter of the balloon when mounted on a catheter in its deflated and wrapped condition or the smallest hole through which the deflated wrapped balloon catheter can pass.

Balloon Compliance—refers to change in balloon diameter as a function of inflation pressure.

In various embodiments, when inflated, the one or more balloons may have a shape such as, but not limited to, conical, square, spherical, elliptical, conical-square, long conical-square, conical-spherical, long spherical, tapered, dog bone, stepped, offset, conical-offset, spiral, double-helix spiral, star-burst, split star-bust and trident.

Again, in various embodiments, an end (distal and/or proximal) of the one or more balloons may have a shape such as, but not limited to, conical sharp corner, conical radius corner, square end, spherical end and offset neck.

Table 2 provides a comparison of a plurality of balloons made with various materials:

TABLE 2

| | | | | | Max. Rated | | |
|---|---|---|---|---|---|---|---|
| | | | | | Pressure | | |
| | Tensile | | | | for PTCA* | | Sterilization |
| Materials | Strength | Compliance | Stiffness | Profile | ATM | PSI | Methods |
| PET | High- | Low- | High | Low | 20 | 294 | EtO or |
| | Very | Medium | | | | | Radiation |
| | High | | | | | | |
| Nylons | Medium- | Medium | Medium | Low- | 16 | 235 | EtO |
| | High | | | Medium | | | |
| PE | Low | High | Low | High | 10 | 147 | EtO or |
| (crosslinked) | | | | | | | Radiation |
| and other | | | | | | | |
| polyolefins | | | | | | | |
| Polyurethanes | Low- | Low- | Low- | Medium- | 10 | 147 | EtO |
| | Medium | High | Medium | High | | | |
| PVC | Low | High | Low | High | 6-8 | 88-117 | Radiation |
| (flexible) | | | | | | | |

*The maximum rated pressure is based on practical limitations and usefulness

Material of the balloon is an important factor. If the softening temperature (Tg) of a balloon's material is too low, the balloon may deform during use when exposed to vapor or steam. For example, the Tg of PET is 75° C. This means that after just one use, the PET balloon may deform and may not be useable for conducting additional ablation shots of a given vein or of other veins in the patient. Therefore, it is desirable to use a material that has a Tg greater than 100° C. to be functional. In embodiments, there are two balloons, where each balloon has a different Tg value. In embodiments, the Tg value of each balloon is within a range of 60° C. to about 200° C. In some embodiments, the Tg is 80° C. In some embodiments, the Tg is 150° C. In some embodiments, the outer balloon is composed of Pellethane®.

It is also desirable to use a material that has a sufficiently wide elasticity range at various operating temperatures. If the elasticity range is too low, the yield point is passed during operation and the balloon deforms such that the ablation zone may not be properly positioned during operation.

In embodiments, material of the inner balloon is non-compliant to semi-compliant, implying that the material eliminates any folds that may have been present during packaging, and conforms to the target anatomy for better contact. A compliant balloon is likely to have a fixed volume at a fixed pressure. It is desirable that material of the inner balloon is more rigid than that of the outer balloon, so as to maintain a certain shape. Some of the semi-compliant balloon materials, for example PEBA families, face mechanical and thermal challenges when introduced to steam. Therefore, a preferred balloon material is a copolymer called Arnitel. Arnitel is also relatively semi-compliant but has higher softening and melt temperatures versus standard PEBA polymers. Materials such as Arnitel may be used to make the inner balloon, outer balloon, and shaft applications, in accordance with the embodiments of the present specification. An advantage of using it as a shaft material is that it is thermally bondable with inner balloons currently made using PET, thereby eliminating the need to use an adhesive bonding process.

Ablation catheter needs to be sheathed and unsheathed, particularly if the ablation catheter is only 1 fr or less than the guide sheath. In some embodiments, a hydrophilic coating on the ablation catheter is used to enable easy sheathing. Coating also enables efficient energy transfer to and protects the outer balloon surface from charring. In embodiments, the balloons are pleated in a specific direction, such as in the rightward direction, to allow for easy sheathing/re-sheathing.

In some embodiments, the guide sheath is braided and is of a higher durometer than the ablation catheter. Guide wires are typically positioned in the catheter or outer catheter sheath lining to help bend the sides. A distal opening of the guide sheath is positioned normal to the opening of the vein. In some embodiments, two guide sheaths are provided, where the two guide sheaths are of two different radii and of different deflection characteristics. In an embodiment, one pull wire for the first sheath creates a radius in a range of 0.1 inches to 0.75 inches. In an embodiment, a second pull wire to the second sheath creates a radius in a range of 0.5 inches to 10 inches. In some embodiments, catheter deflection is performed via a handle actuator. Each pull wire is attached to a knob or lever in the handle. A user would twist the knob or pull the lever to apply tension to the tip and deflect. The radius is determined by the catheter construction. In embodiments, each half of the catheter has a separate and unique construction, allowing for the two unique radii.

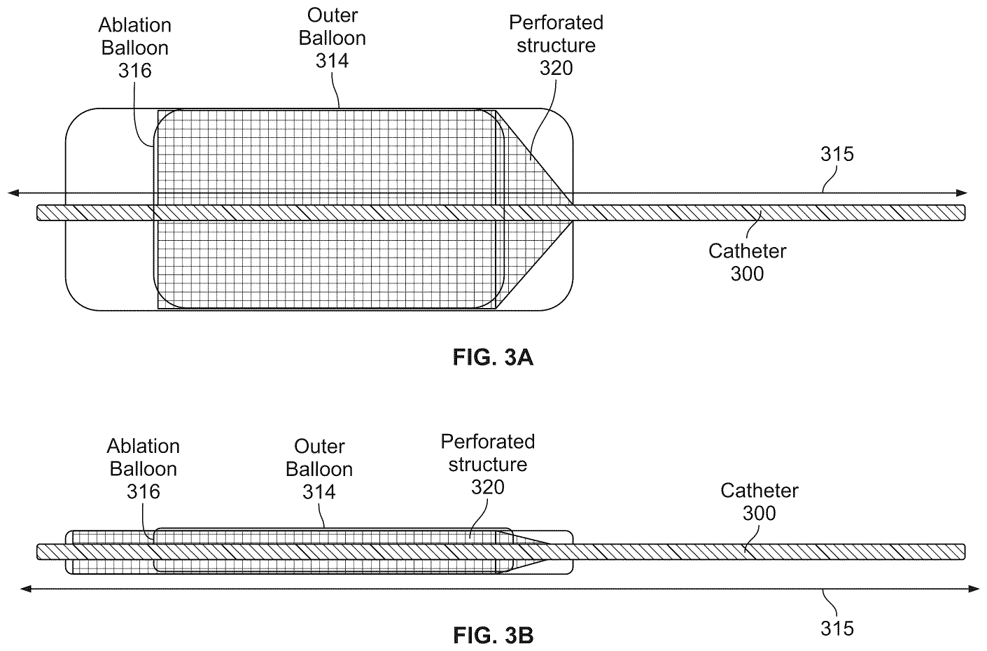

FIG. 3A illustrates a distal portion of a catheter 300 (catheter 101 of FIG. 1A) with an outer balloon 314 (outer balloon 114 of FIG. 1A), a perforated layer 320 (perforated layer 120 of FIG. 1) and an ablation balloon 316 (ablation balloon 116 of FIG. 1A), in an expanded configuration. FIG. 3B illustrates the distal portion of catheter 300 with outer balloon 314, perforated layer 320 and ablation balloon 316, in a compressed configuration. In an embodiment, the perforated layer 320 may be made of self-expanding Nitinol material, that expands as the ablation balloon 316 is inflated and contracts as the outer balloon 314 is deflated. When all elements are in the second state, or expanded configuration, the perforated structure 320 is positioned such that an outer surface of the perforated structure 320 contacts an inner surface of the outer balloon 314 and an inner surface of the perforated structure 320 contacts an outer surface of the ablation balloon 316. These areas of contacts limit the transfer of ablative energy from the ablation balloon 316 to the outer balloon 314 and result in zones of subtherapeutic ablation in the target tissue.

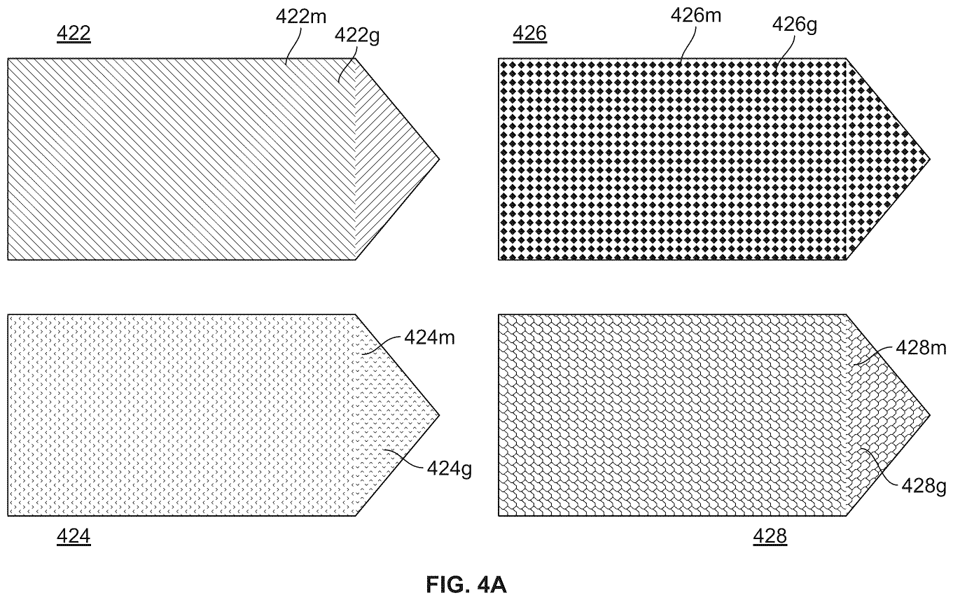

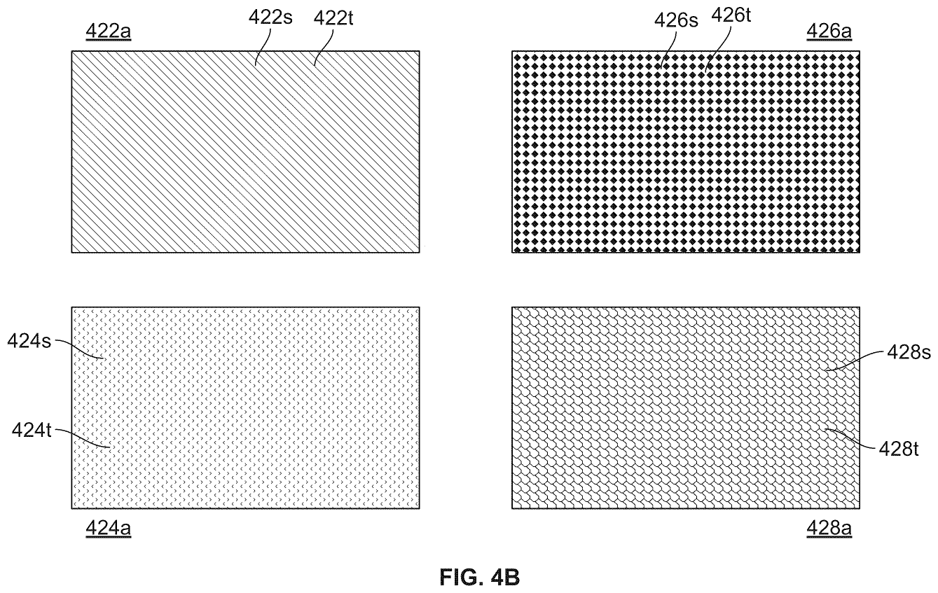

The shape and the dimensions of perforated layer 320 defines the shape and dimensions of the therapeutic and subtherapeutic ablation zones created as a result of an ablation procedure. FIG. 4A illustrates at least four different embodiments 422, 424, 426 and 428, of the sheet used to construct perforated layer 320. The sheet may be made from a metal such as stainless steel (SST), NiTi, or Chromium, or other materials, such as, but not limited to, thermoplastic, silicone, or polyimide aerogel. The different embodiments 422, 424, 426 and 428 enable different patterns of therapeutic and subtherapeutic ablation zones to be created through said intervening structure. Each embodiment 422, 424, 426, 428 comprises areas of the sheet comprising the material 422m, 424m, 426m, 428m and gaps 422g, 424g, 426g, 428g between the areas of material. The gaps 422g, 424g, 426g, 428g permit the full transfer of ablative energy from the ablation balloon to the outer balloon and target tissue for therapeutic ablation while the areas with material 422m, 424m, 426m, 428m limit the transfer of ablative energy from the ablation balloon to the outer balloon and target tissue for subtherapeutic ablation.

FIG. 4B illustrates corresponding ablation patterns formed as a result of selecting each one of embodiments shown in FIG. 4A. Therefore, ablation pattern 422a is formed by the use of embodiment 422, ablation pattern 424a is formed by the use of embodiment 424, ablation pattern 426a is formed by the use of embodiment 426, and ablation pattern 428a is formed by the use of embodiment 428. In each pattern 422a, 424a, 426a, and 428a, areas 422s, 424s, 426s, 428s (dark areas) represent areas of subtherapeutic ablation (where the material of the perforated layer blocked some of the ablative energy), while areas 422t, 424t, 426t, 428t (lighter areas) represent areas of therapeutic ablation (where the gaps of the perforated layer allowed for the full transfer of ablative energy). A third zone can be referred to as the non-ablation zone, which includes areas around the sub-therapeutic and the therapeutic ablation zones, and where effects of ablation are not present. Non-ablation zones, such as zone 126 in FIG. 1, may also be referred to as insulation zones, wherein each of the one or more non-ablation zones is defined by a surface area of the outer balloon that is proximal or distal to sub-therapeutic and therapeutic ablation zones and wherein each of the one or more non-ablation zones has an average temperature that is less than an average temperature of the sub-therapeutic and therapeutic ablation zones.

In some embodiments, referring again to FIG. 3, an entire surface of the catheter 300 is coated with heparin. In some embodiments, the ablation balloon 316 is movable along a longitudinal axis 315 of the balloons 314, 316 within and along an entire length of the outer balloon 314, to better position inner ablation balloon 316 inside outer balloon 314, using a wire mechanism in a handle at the proximal end of catheter 300. In embodiments, the perforated layer 320 expands with the inflation of the ablation balloon 316 and contracts with the deflation of the outer balloon 314, shortening in length during the expansion phase, wherein a distal end of the perforated layer 320 is pulled back as a proximal end the perforated layer 320 is fixed and immovable.

In embodiments, at least one dimension of inner ablation balloon 316 is different than the outer balloon 314 by at least 10%. In some embodiments, the dimension is a length of ablation balloon 316. In embodiments, a shape of ablation balloon 316 is different from that of the outer balloon 314. In embodiments, an intersection of the shapes of ablation balloon 316 and outer balloon 314 determine the shape and/or size of an ablation zone (including the therapeutic and the sub-therapeutic ablation zones).

Referring again to FIG. 1A, in some embodiments, one or more sensors 118m are optionally attached to the distal end of body 104 distal to outer balloon 114/314. In embodiments, the sensors are configured to determine either temperature, pressure or electrical impedance for measuring tissue signals and aiding in monitoring/controlling the ablation process. In some embodiments, one or more sensors in the ablation zone are utilized to monitor contact of the inner and outer balloons. In embodiments, the temperature of the inner balloon is monitored. During and after ablation, the temperature and pressure in the outer balloon is monitored to maintain at desired therapeutic values.

Once both balloons 114, 116 are inflated, a length of outer balloon 114 is greater than a length of inner ablation balloon 116 and a diameter of ablation balloon 116 approximates a diameter of outer balloon 114. Catheter 101 also includes at least one flexible heating chamber 102 proximate to a proximal end of outer balloon 114. In one embodiment, as shown in FIG. 1A, two heating chambers 102 are arranged in series in catheter body 104, particularly in second lumen 108. An RF generator 122 is coupled to a plurality of electrodes (such as, electrodes 208, 212 of FIG. 2B) included within the heating chamber 204.

During use, the first fluid pump 110 supplies air or CO₂ or another cooling/insulating fluid via the first lumen 106 causing outer balloon 114 to inflate, water or saline pump 112 supplies water/saline to a proximal end of heating chambers 204 via water/vapor lumen 108 while RF generator 122 supplies electrical current to the electrodes, causing them to heat up and vaporize water/saline flowing through heating chambers 204. The generated hot fluid or vapor exits through infusion ports 118, thereby inflating inner ablation balloon 116 such that ablation balloon 116 comes into contact with outer balloon 114 and outer balloon 114 comes into contact with the target tissue proximate the equators of both balloons 114, 116. This creates a hot zone or ablation zone 124 proximate the equator of the outer balloon 114. Ablation zone 124 comprises therapeutic zones 124t, where the gaps (for example, 422g in FIG. 4A) of the perforated structure 120 are located, and subtherapeutic zones 124s, where the material (for example, 422m of FIG. 4A) of the perforated structure 120 is located. Non-ablation zones 126 are located on outer balloon 114 where inflated ablation balloon 116 is not in contact with inflated outer balloon 114. Heat is transferred from inside inner ablation balloon 116 through outer balloon 114 at ablation zone 124 and into the tissue to ablate the tissue. The flexible heating chambers 204 impart improved flexibility and maneuverability to catheter 100, allowing a physician to better position catheter 100 when performing any ablation procedures, such as that of ablating an arrhythmia focus in a heart of a patient.

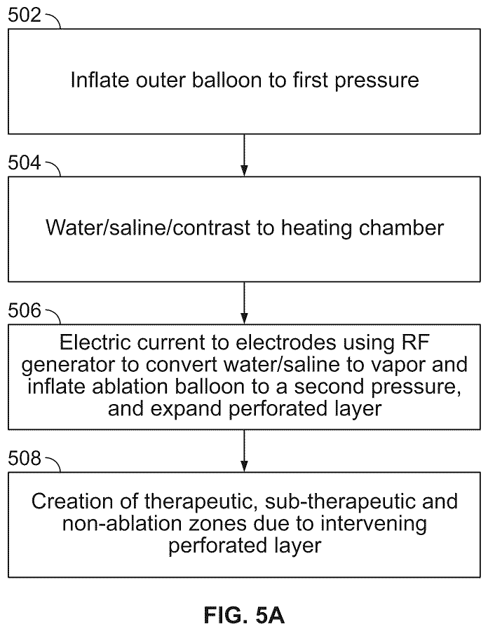

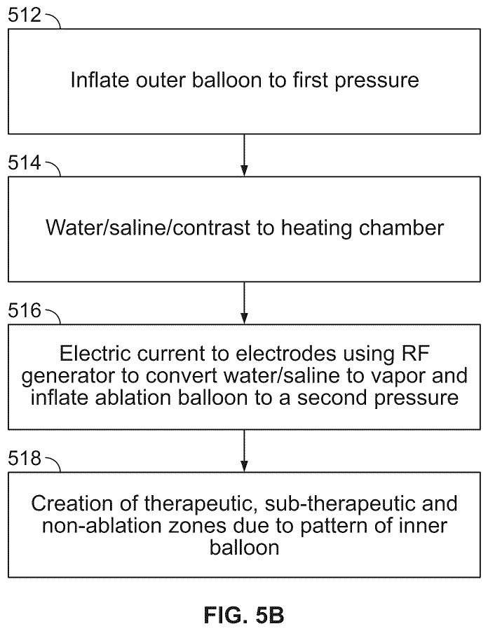

FIG. 5A is a flowchart illustrating the steps involved in one embodiment of a method of using system 100 of FIG. 1A or 1B to ablate target tissue. At step 502, the outer balloon is inflated to a first pressure (P1) using air, CO₂, or other fluid with or without radiopharmaceutical contrast by operating a first fluid pump. A target area is optionally mapped using the outer balloon. At step 504, water, saline, and/or contrast is provided to the at least one heating chamber by operating a second fluid pump. At step 506, electric current is provided to electrodes of the heating chamber, using the RF generator, to convert water/saline to hot fluid or vapor that exits the infusion ports to inflate the ablation balloon to a second pressure (P2) greater than or equal to P1 thereby causing the inner ablation balloon to contact the outer balloon. At this step, perforated layer also expands with the inflation of the inner ablation balloon, so that the perforated layer intervenes between the inner ablation balloon and the outer balloon. At step 508, a pattern of subtherapeutic and therapeutic ablation zones are created on the anterior or distal surface of the outer balloon, based on the pattern of the perforated layer, where the ablation balloon is touching the outer balloon through the perforated layer, and where the outer balloon is touching the target tissue, while non-ablation zones exist where the ablation balloon is not touching the outer balloon through the intervening perforated layer. Electrical activity is optionally monitored during and after the ablation process to document complete ablation of the target area. Optionally, mapping electrodes monitor tissue impedance and tissue temperature to guide the ablation. Additionally, optionally, pacing distal to the balloon is performed to check for adequacy of ablation. The inner balloon can be optionally pre-inflated with CO₂ or air to a pressure less than or equal to P2 prior to insufflating it with vapor.

FIG. 5B is a flowchart illustrating the steps involved in one embodiment of a method of using the ablation system of FIG. 2C to ablate a body tissue. At step 512, the outer balloon is inflated to a first pressure (P1) using air, CO₂, or other fluid with or without radiopharmaceutical contrast by operating a first fluid pump. A target area is optionally mapped using the outer balloon. At step 514, water, saline, and/or contrast is provided to the semi-porous thermal chamber by operating a second fluid pump. At step 516, electric current is provided to electrodes of the semi-porous thermal chamber, using the RF generator, to convert water/saline to hot fluid or vapor that exits the infusion ports to inflate the ablation balloon to a second pressure (P2) greater than or equal to P1 thereby causing the inner ablation balloon to contact the outer balloon. At step 518, a pattern of subtherapeutic and therapeutic ablation zones are created on the anterior or distal surface of the outer balloon, based on a pattern of the inner balloon, where the inner balloon is touching the outer balloon only at certain portions, and where the outer balloon is touching the target tissue, while non-ablation zones exist where the inner balloon is not touching the outer balloon. Electrical activity is optionally monitored during and after the ablation process to document complete ablation of the target area. Optionally, mapping electrodes monitor tissue impedance and tissue temperature to guide the ablation. Additionally, optionally, pacing distal to the balloon is performed to check for adequacy of ablation. The inner balloon can be optionally pre-inflated with CO₂ or air to a pressure less than or equal to P2 prior to insufflating it with vapor.

In various embodiments, the outer balloon comprises silicone. In various embodiments, the inner balloon comprises med-durometer urethane, PET, or Pebax. In various embodiments, the inner balloon comprises a material configured to be semi-compliant while also heat tolerant.

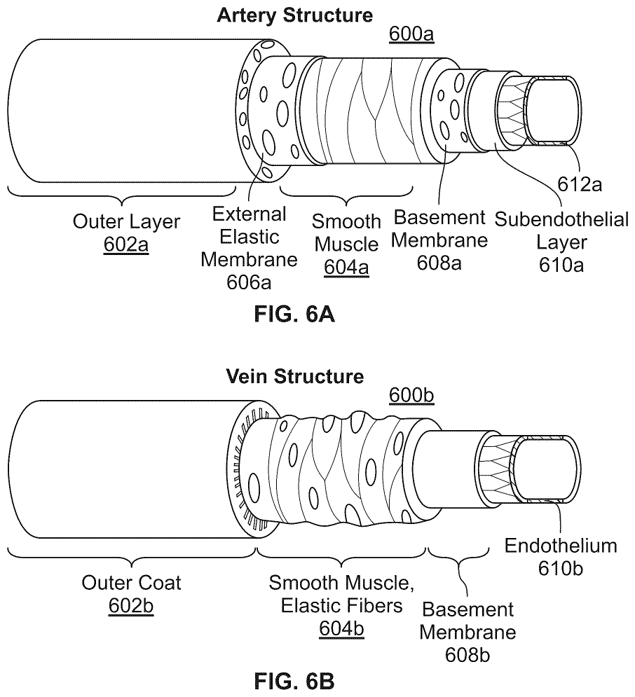

Embodiments of the present specification are described in reference to tissue ablation within an artery or a vein. Particularly, to contextualize the ablation method of FIGS. 5A and 5B, the vascular anatomy is shown in FIG. 6A-6B. FIG. 6A illustrates a known structure of an artery. An artery 600a comprises an outermost elongated cylindrical layer 602a that encapsulates a smooth muscle 604a which is wrapped inside a coating of an external elastic membrane 606a. Smooth muscle 604a forms a covering layer for a basement membrane 608a, inside which is a subendothelial layer 610a and an endothelium 612a, where the latter is hollow for passage of blood away from the heart to the rest of the body.

FIG. 6B illustrates a known structure of a vein. A vein 600b comprises an outermost elongated cylindrical layer 602b that encapsulates a smooth muscle comprising elastic fibers 604b. Smooth muscle 604b forms a covering layer for a basement membrane 608b, inside which is an endothelium layer 610b, where the latter is hollow for passage of blood towards the heart from the rest of the body. Often one or more valves inside the inner layer of endothelium layer 610b regulate the flow of blood through the veins.

### Application in Renal Denervation

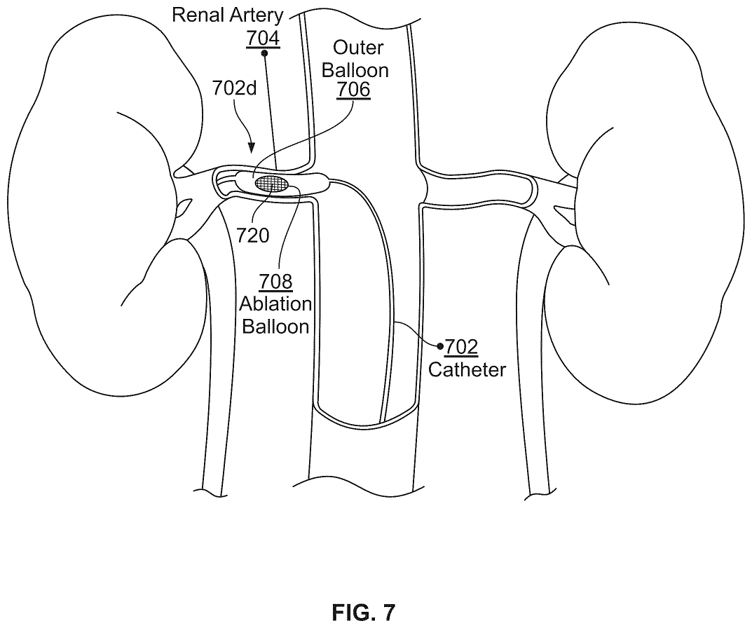

Patients with cardiovascular conditions, such as and not limited to hypertension, can be treated through renal denervation using the systems and methods of the embodiments of the present specification. Embodiments of the present specification are now explained with reference to application in renal denervation. FIG. 7 illustrates a positioning of a distal portion 702d of an ablation catheter 702 within a renal artery 704 in accordance with embodiments of the present specification. The figure shows an inflated outer balloon 706 at the distal portion 702d of catheter 702. Outer balloon 706 is inflated and contacts an inner surface of the renal artery 704 while an inner ablation balloon 708 is expanded with heated vapor. The heat generated within ablation balloon 708 permeates through a perforation layer 720 that is positioned between ablation balloon 708 and outer balloon 706, to create areas of subtherapeutic and therapeutic ablation in the target tissue within renal artery 704.

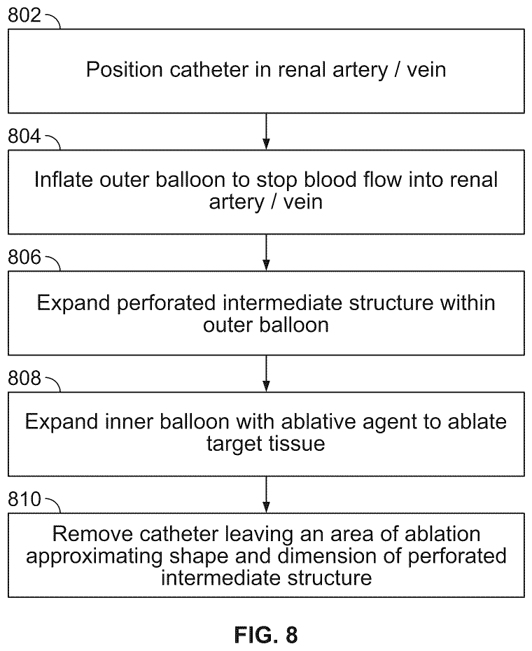

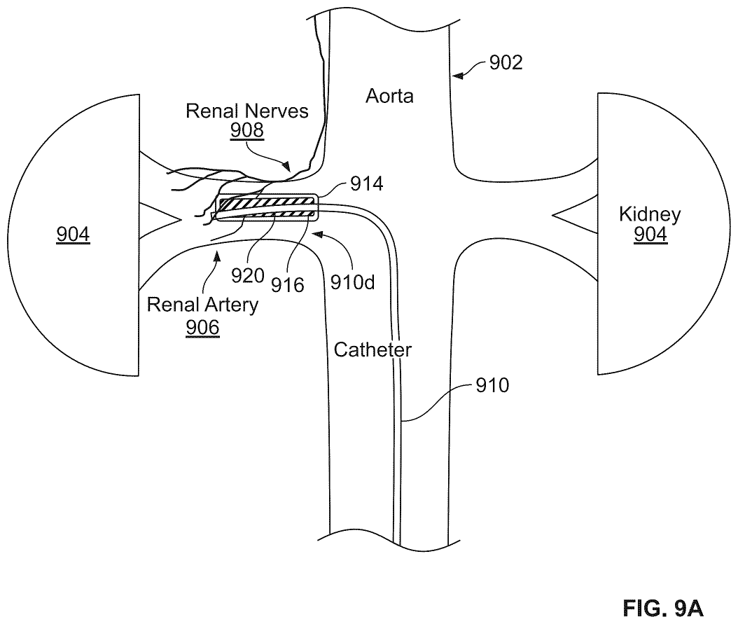

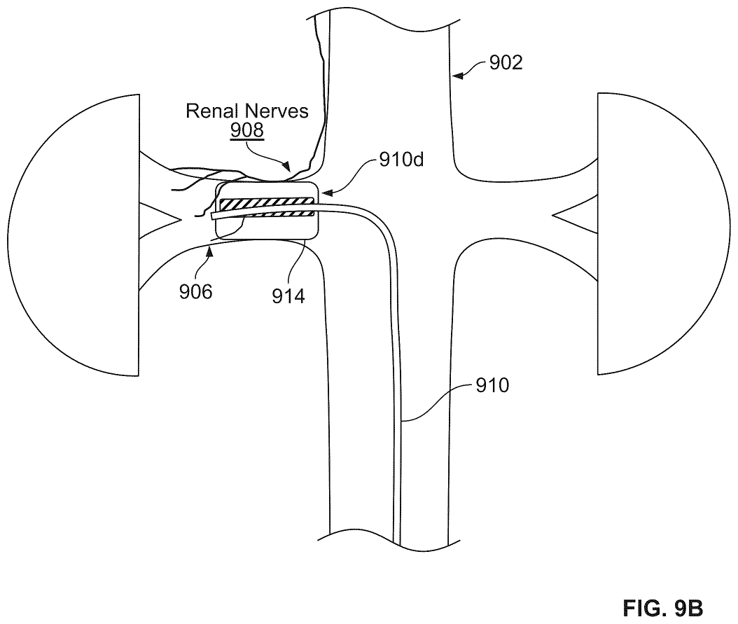

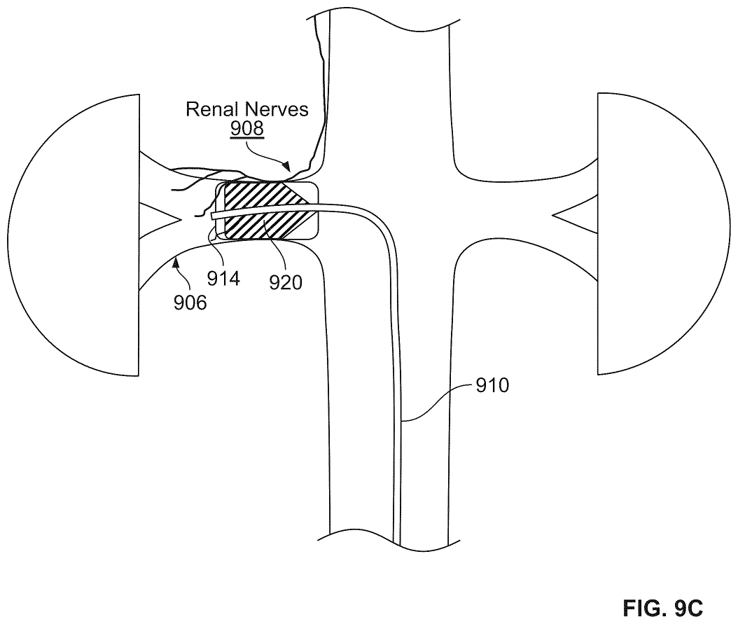

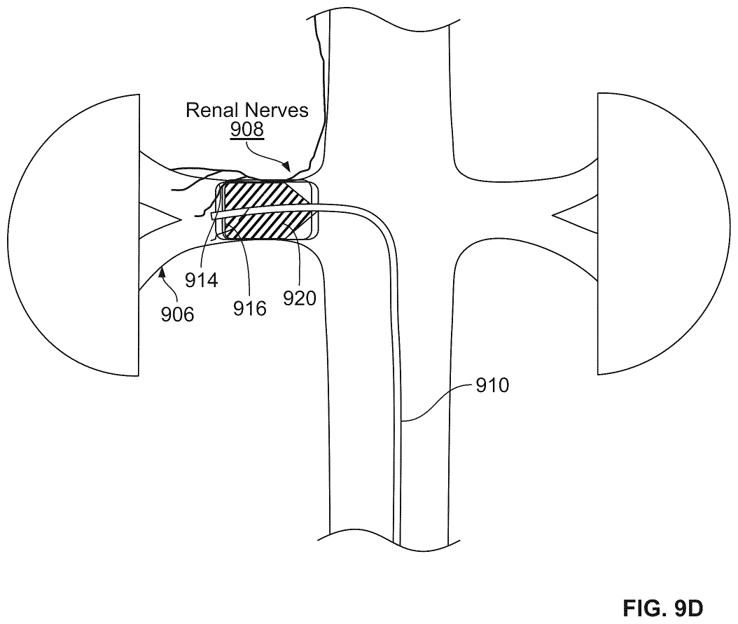

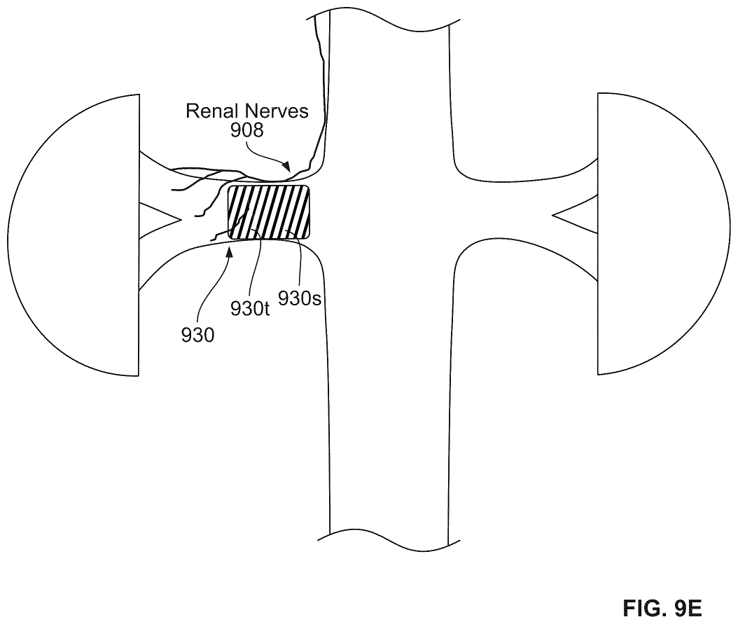

FIG. 8 is a flow chart illustrating an exemplary process for renal denervation in accordance with some embodiments of the present specification. At step 802, catheter is positioned through an aorta between the kidneys, and pushed further till distal end of the catheter is positioned in a renal artery or a renal vein of a patient. FIG. 9A illustrates a catheter 910 passed through an aorta 902 between kidneys 904. Aorta 902 branches out through arteries 906 towards kidneys 904. Renal nerves 908 are also shown that regulate the flow of blood from kidneys 904. A catheter 910 is shown positioned through aorta 902 such that distal end 910d of catheter 910 is positioned within renal artery 906 or proximate renal nerves 908. A portion of distal end 910d comprises an outer balloon 914, a perforated layer 920, and an inner ablation balloon 916 in their compressed states.

FIG. 9B illustrates catheter 910 of FIG. 9A with expanded configuration of an outer balloon 914. Referring simultaneously to FIGS. 8 and 9B, at step 804, outer balloon 914 is inflated so that it expands within artery 906 to a sufficiently large volume so as to stop the flow of blood into artery 906. Alternatively, the catheter is positioned within a renal vein and outer balloon 914 is expanded to block flow of blood through the target renal vein. In some embodiments, outer balloon 914 is expanded or inflated with air such as carbon dioxide (CO₂) or by using an insulative fluid. In some embodiments, the outer balloon is inflated to a pressure in a range of 0.1 to 10 psi.

FIG. 9C illustrates catheter 910 of FIG. 9B with expanded configuration of a perforated layer 920. Referring simultaneously to FIGS. 8 and 9C, at step 806, perforated layer 920 is expanded within outer balloon 914, to a volume so that expanded perforated layer 920 occupies the interior volume of outer balloon 914. In an embodiment, the perforated layer 920 may be made of self-expanding Nitinol material, that expands as the an inner ablation balloon 916 is inflated and contracts as the outer balloon 914 is deflated. In embodiments, outer surface of expanded perforated layer 916 is in contact with at least a portion of internal surface of expanded outer balloon 914.

FIG. 9D illustrates catheter 910 of FIG. 9C with expanded configuration of an inner ablation balloon 916. Referring simultaneously to FIGS. 8 and 9D, at step 808, inner ablation balloon 916 is expanded. In some embodiments, inner ablation balloon 916 is expanded with an ablative agent, such as saline that is heated by passing the saline over a heating chamber, and converted to heated vapor. A physician or any other person operating the system in accordance with the present specification, may trigger inflation of the inner balloon by pressing a switch, a button, or a pedal. The trigger may first activate a pump that pushes saline through tubing and into the ablation catheter. The amount of saline pushed is sufficient to first wet the electrodes but insufficient to fill inner balloon 916. The RF electrodes are then activated to heat the saline. Steam or vapor enters inner balloon 916. Inner balloon 916 inflates, due to the influx of steam or vapor, and forms a specifically defined ablation zone with outer balloon 914, through perforated layer 920.

In some embodiments, the inner balloon is inflated to a pressure in a range of 0.5 to 20 psi, and preferably within a range of 2.5 to 3.5 psi. In embodiments, the pressure within the inner balloon is always greater than that of the outer balloon during the step of ablation. In its expanded configuration, outer surface of inner ablation balloon 916 is in contact with outer balloon 914 through the gaps or spaces in perforated layer 920. Heated vapor generated within inner ablation balloon 916 creates areas of therapeutic ablation where outer surface of inner ablation balloon 916 in in contact with inner surface of outer balloon 914 (at gaps of perforated layer 920), creates areas of sub-therapeutic ablation where the material areas of the perforated layer are positioned between areas of contact of the inner ablation balloon 916 with the outer balloon 914, and creates non-ablation zones where there is no contact between the inner balloon 916 and the outer balloon 914. In embodiments, outer balloon 914 is greater in volume to inner balloon 918 by 3 to 75 cc, and an air gap is present on the distal and proximal ends, or additionally at various locations along the length of outer balloon 914 based on the shape and size of inflated inner balloon 918, between outer balloon 914 and inner balloon 916. The air gap provides for an insulation zone. The insulation zone where inner ablation balloon 916 does not contact either outer balloon 914 or perforated layer 920, is also referred to herein as the area of non-ablation.

Steam or vapor is produced for a pre-defined period of time. The time period may be pre-defined to be in a range from a few seconds to a few minutes. In some embodiments, the range is from 5 seconds to two minutes. In one embodiment, the time period is defined to be of 20 seconds. After the pre-defined period of time, the system shuts off and stops the generation and supply of steam. Once therapeutic, safety, or time endpoint is reached, first the electrodes are shut off and then saline is shut off. In some embodiments, a power of 60 W is used by the generator to operate the steam and supply the saline. In another embodiment a power of 120 W is used. The wattage may be increased or modified depending on the electrode. In some embodiments, the energy delivered by the steam may be controlled based on the flow of the saline. As a result, if the flow rate of saline is low, the energy level is adjusted and lowered to prevent low impedance faults.

The saline flow rate may also depend on size of the electrode, including the length, width, and periphery of the electrode, and the power supplied. Generation of steam or vapor results in transfer of heat energy from inner balloon 916 through perforated layer 920 to outer balloon 914 to ablate the target renal artery and/or vein. In some embodiments, contact of the inner and outer balloons at the ablation zone is documented and confirmed using fluoroscopy, 3D mapping and/or endoscopy. In some embodiments, one or more sensors in the ablation zone are utilized to monitor contact of the inner and outer balloons. In embodiments, the temperature of the inner balloon is monitored. During and after ablation, the temperature and pressure in the outer balloon is monitored to maintain desired therapeutic values.

Once the ablation treatment process is performed, electrodes/saline are shut off, resulting in an almost instant collapse of inner balloon 916. In some embodiments, at this point CO₂ may be automatically injected to maintain a volume of or a pressure in outer balloon 914. Volume of outer balloon 914 is maintained so that the ablation step is repeated (if required) wherein heat is delivered for a second duration. In some embodiments, the second duration ranges between 50% and 250% of the first duration. In one embodiment, inner balloon 916 is configured to decrease in volume, without applying negative pressure, after terminating RF energy to the electrodes/or flow of saline. In one embodiment, outer balloon 914 is configured to automatically receive an input of fluid (such as CO₂) after terminating electrodes/saline without measuring for pressure, temperature, or volume changes.

FIG. 9E shows the areas of therapeutic and sub-therapeutic ablation in a pattern 930 that is based on the pattern of perforated layer 920, after ablation is performed. Referring simultaneously to FIGS. 8 and 9E, at step 810, catheter 910 is removed from the renal artery 906 (or from proximity of target renal nerve 908). The ablation pattern 930 includes areas of subtherapeutic ablation 930s and therapeutic ablation 930t in the target ablation region. Pattern 930 approximates the shape and the dimension of perforated layer 920 with areas of therapeutic ablation 930t shown in grey (light areas within pattern 930) and areas of subtherapeutic ablation 930s shown in black (dark areas within pattern 930) with the intent of causing a pattern. Therefore, embodiments of the present specification achieve deep ablation without large contiguous area of deep ablation to prevent large contiguous area of collagen deposition, and as a result prevent stricture formation.

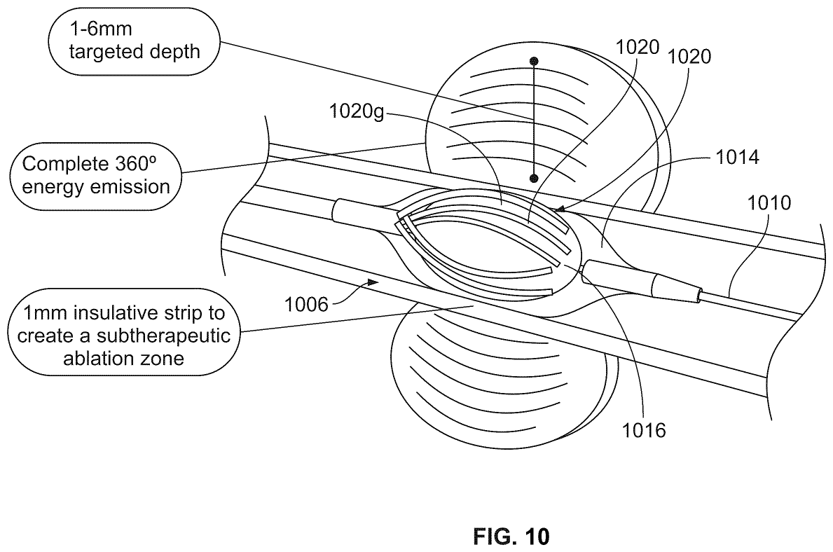

FIG. 10 illustrates an example of geometry of ablation using the ablation systems of the present specification. The figure shows the position of a distal end of a catheter 1010 inside an artery 1006 (or vein). The figure further shows an outer balloon 1014 in its expanded configuration and the inner ablation balloon 1016 also in its expanded configuration. In most embodiments, the amount of heat generated during the ablation is proportional to one or more parameters that can be controlled by the user operating the ablation device. The parameters include, and are not limited to, power, current, voltage, impedance, and time or duration of control of said parameter(s). The amount of heat generated during ablation directly impacts the depth of ablation achieved within the target tissue inside artery 1006 (or wall of vein—not shown). In embodiments, the depth of ablation achieved ranges from 1 to 6 millimeters (mm). In the pictured embodiment, elongate strips 1020 of material are positioned between the inner balloon 1016 and the outer balloon 1014 with gaps 1020g between the strips, providing insulating regions to limit the transfer of ablative energy from the inner ablation balloon 1016 and creating subtherapeutic ablation zones. In embodiments, the strips range from 0.1 mm to 3 mm wide. In some embodiments, the strips are 1 mm wide.

The insulative strips 1020 create subtherapeutic or non-ablative areas, thereby limiting a cumulative circumferential width of ablation of the vascular media or adventitia to be less than 50%. Even though the ablative energy emitted from within inner ablation balloon 1016 extends circumferentially all around shaft of catheter 1010, causing a 360° emission of energy either the elongate strips 1020 break the ablative energy into linear strips to create ablated and non-ablated vessel, thereby limiting a cumulative circumferential width of ablation of the vascular media or adventitia to be less than 50%.

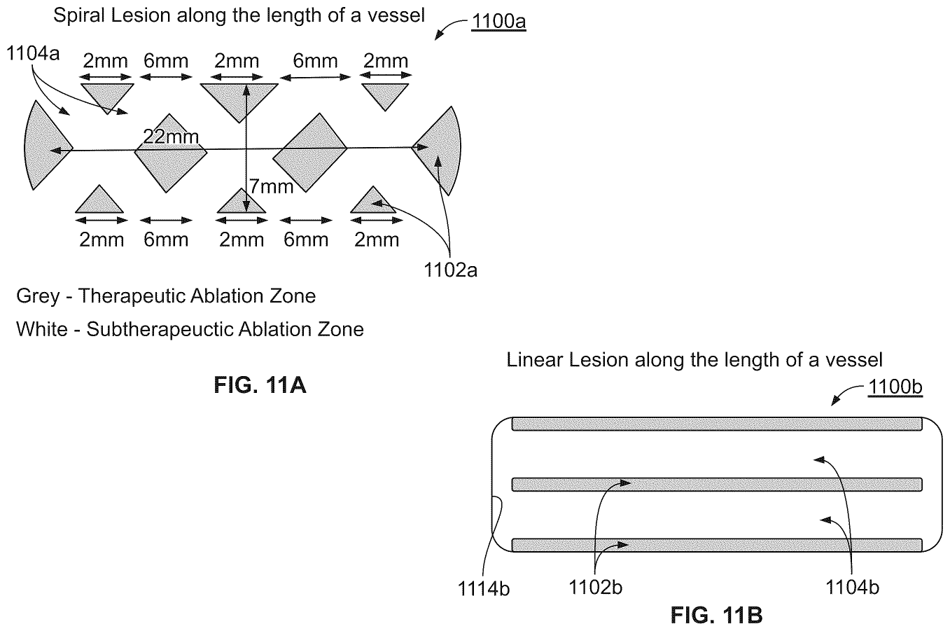

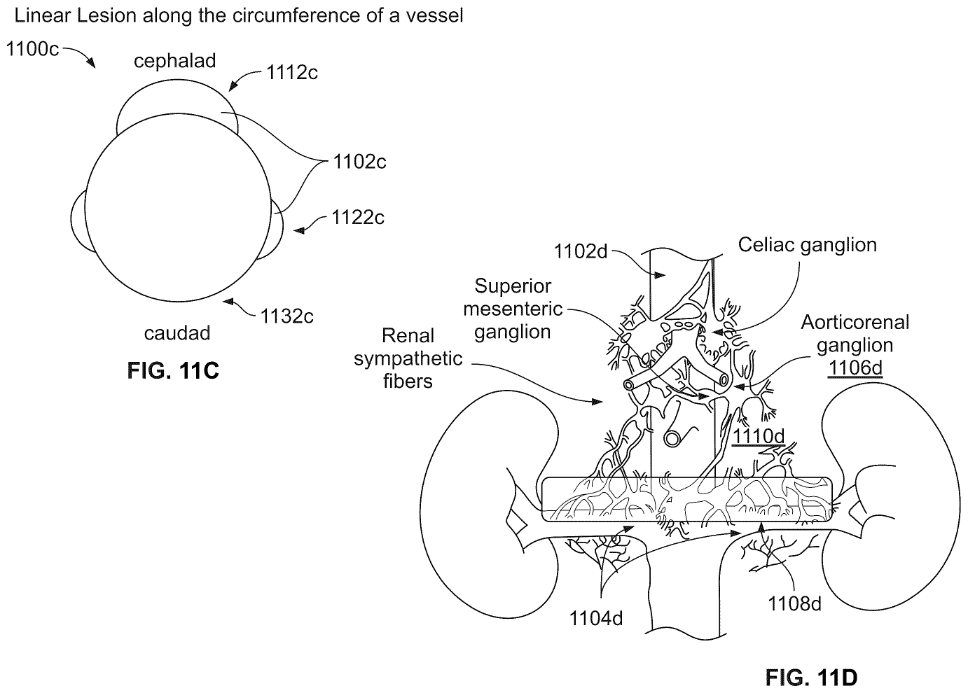

The geometry of ablation, which refers to the three-dimensional shape of therapeutic and sub-therapeutic ablation zones formed by the methods and systems of the present specification, can be circular or non-circular. In some embodiments, the geometry of ablation is linear along the length of an artery, such as a renal artery. In most cases, renal arteries have a length in a range from 4 to 6 cm and a diameter that approximates 5 to 6 mm. Inner ablation balloon configurations can be varied to achieve a particular type of ablation geometry that can be based on the numbers, sizes, and locations of target tissues for ablation, within the renal artery. Linear ablation is achieved along the length of the renal artery. In some embodiments, spiral ablation is achieved, such as for example, shown in FIG. 11A. It should be noted that the term ‘ablation’ herein refers to areas of therapeutic and sub-therapeutic ablation.

FIG. 11A illustrates an exemplary spiral ablation geometry 1100a achieved in accordance with some embodiments of the present specification. Geometry 1100a is achieved from left to right along the length of renal artery. Grey areas 1102a of geometry 1100a indicate the areas of therapeutic ablation (where the gaps of the perforated layer were positioned between the inner ablation balloon and the outer balloon), and the non-grey areas 1104a represent areas of subtherapeutic ablation (where the material of the perforated layer was positioned between the inner ablation balloon and the outer balloon). The actual ablation geometry depends on the inner surface of the renal artery, shape and geometry of the inner ablation balloon and outer balloon, and shape of the perforated layer. Geometry 1100a is achieved with a specific shape and geometry of the inner ablation balloon, such as but not limited to starburst shape, spiral shape or mesh shape, which leaves subtherapeutic ablation zones of approximately 2 mm spaced between therapeutic ablation zones of approximately 6 mm, along the length of the artery. In some embodiments, the diameter of the circumferential subtherapeutic ablation and therapeutic ablation zones is approximately 7 mm and/or is the same as the diameter of the renal artery. Total length of the subtherapeutic ablation and therapeutic ablation zones can extend to approximately 22 mm.

FIG. 11B illustrates an exemplary linear ablation geometry 1100b achieved in accordance with some embodiments of the present specification. Geometry 1100b is achieved longitudinally along the length of the renal artery in the form of parallel therapeutic ablation zones 1102b (where the gaps of the perforated layer were positioned between the inner ablation balloon and the outer balloon) spaced from each other with parallel subtherapeutic ablation zones (where the material of the perforated layer was positioned between the inner ablation balloon and the outer balloon) positioned between. A line 1114b illustrates possible border of an outer balloon within which ablation zones 1102b, 1104b are formed by the inner ablation balloon.

In some embodiments, ablation has a geographical pattern of distribution wherein the thermal ablation follows the pattern of distribution of renal nerves. In yet some embodiments, ablation has a geographical pattern wherein the ablation area or volume is more toward the cephalad portion than the caudad portion of the vessel. FIG. 11C illustrates a cross-section view of a linear ablation geometry 1100c showing the ablation area and depth or volume 1102c to be greater towards the cephalad portion 1112c, relatively less in the region between the cephalad and caudad 1122c, and very little or no ablation towards the caudad portion 1132c. FIG. 11D illustrates a sample anatomy 1100d of renal sympathetic fibers 1110d spread over an aorta 1102d and extending across a cephalad region of renal arteries 1104d. The illustration also shows renal nerves extending from aorticorenal ganglion 1106d in the cephalad region. A zone 1108d marked by a rectangular border highlights the shift of ablation zone achieved by some embodiments of the present specification, which is greater in the cephalad region, and which spares the caudad region, therefore selectively affecting the nerves from aorticorenal ganglia 1106d which supply the kidney. The shape of the inner balloon drives the shape of the ablation lesion to optimize the ablation effect specific to the vascular anatomy.

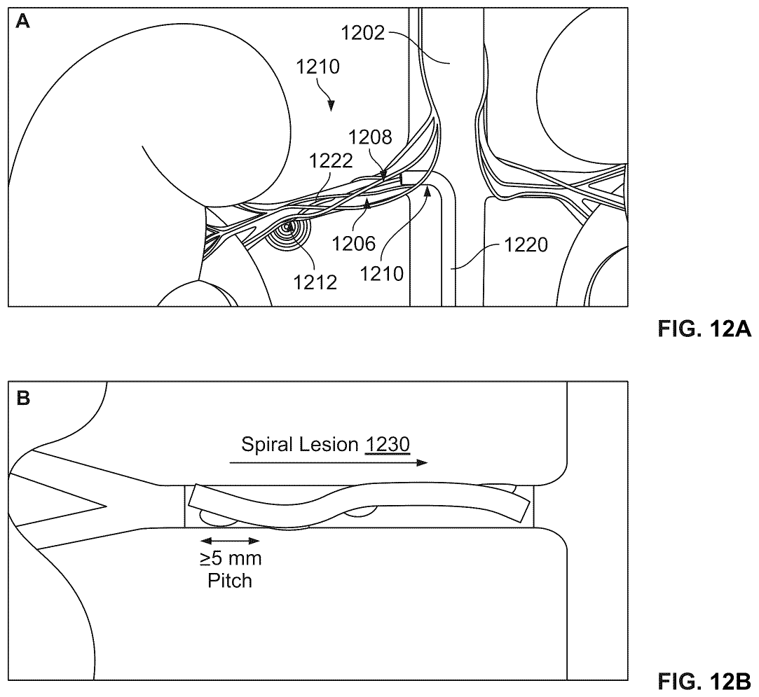

FIG. 12A illustrates the position of distal end 1212 of a catheter 1210 in a renal artery 1206. Catheter 1210 comprises an outer sheath 1220 that is positioned within an aorta 1202. An inner catheter body 1222 is extended from within outer sheath 1220 towards artery 1206, to be positioned for ablation of target tissue inside artery 1206 such as for ablation of renal artery tissue and nerves 1208. A distal portion 1212 of inner catheter body 1222 is positioned proximate to the target tissue for ablation. FIG. 12B illustrates results of ablation using the ablation system of FIG. 12A. A spiral lesion 1230 shown in FIG. 12B is the area that has been ablated. In embodiments, spiral lesion 1230 is created by a spiral inner balloon which creates a spiral ablation lesion. In one embodiment cold fluid is infused through the outer sheath 1220 to further cool the aorta 1202 wall which is not in contact with the ablation balloon.

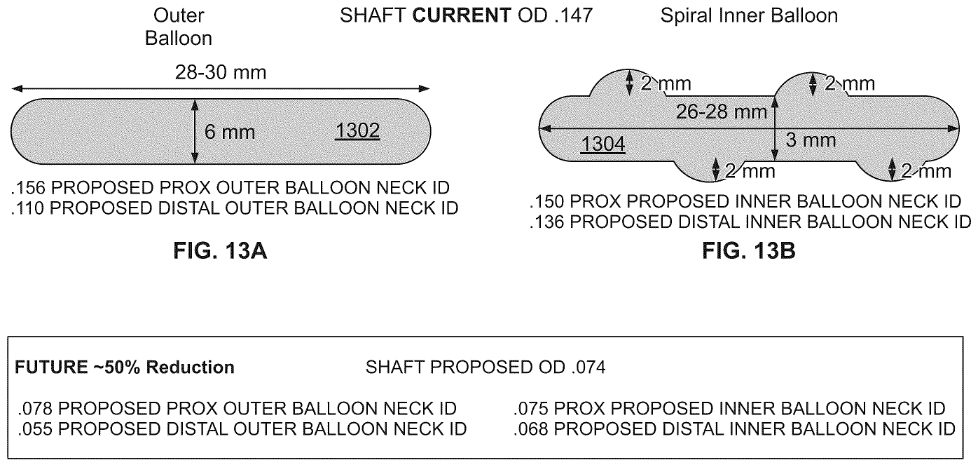

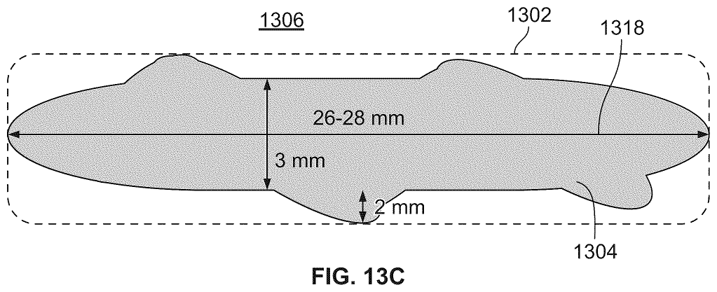

FIG. 13A illustrates an exemplary embodiment of dimensions of an outer balloon 1302 that is used at the distal end of the catheter for ablation of renal artery/vein and/or nerves, in accordance with the present specification. Outer balloon 1302 has a length of approximately 28 to 30 mm and a diameter of approximately 4-9 mm. These dimensions are generally suitable for renal arteries which are between 4 to 6 cm in length and are usually 5 to 6 mm in diameter. In embodiments, the outer balloon has a proximal neck inner diameter ranging from 1 mm to 5 mm and a distal neck inner diameter ranging from 1 mm to 3 mm. In embodiments, the catheter shaft has an outer diameter ranging from 1 to 4 mm.

FIG. 13B illustrates an exemplary embodiment of dimensions of an inner ablation balloon 1304 that is used inside outer balloon 1302 of FIG. 13A, in accordance with embodiments of the present specification. The configuration illustrated in FIG. 13B is preferably used to achieve spiral ablation geometry. A length of balloon 1304 ranges from 26 to 28 mm and an inner diameter of balloon 1304 is approximately 3 mm. A curved tubular extension spirals around the inner diameter of balloon 1304, which extends for another 2 mm in addition to the 3 mm inner diameter. The entire volume inside balloon 1304 is contiguous and is shaped like a spiral tube around a cylindrical tube. In embodiments, the inner balloon has a proximal neck inner diameter ranging from 1 mm to 4 mm and a distal neck inner diameter ranging from 1 mm to 4 mm. In embodiments, the catheter shaft has an outer diameter ranging from 1 to 4 mm.

FIG. 13C illustrates an exemplary configuration 1306 of outer balloon 1302 that can be achieved with inner ablation balloon 1304, in their expanded states and during an ablation process, in accordance with some embodiments of the present specification. In their expanded states, inner ablation balloon 1304 pushes outer linear cylindrical surface of outer balloon 1302 such that a spiral surfaces over and above the linear cylindrical surface of balloon 1302. The dimensions of outer balloon 1302 then proximate the dimensions of expanded inner ablation balloon 1304, as shown. In embodiments, the length of the outer balloon configuration 1306 is in a range of 26-28 mm, with a first inner diameter of 3 mm and a second outer diameter, where the spiral pattern extends outwardly, of 5 mm (spiral extends an additional 2 mm from a central longitudinal axis 1318 of the configuration). The contact point of the two balloon creates the spiral ablation zone.

FIG. 13D illustrates a non-continuous pattern 1360 created by positioning a selectively patterned inner balloon 1363 within an outer balloon 1361, in accordance with some embodiments of the present specification. Similarly to what is described with reference to the intervening or intermediate layer in FIG. 1C, an irregularly shaped inflated inner balloon 1363, for example, spiral or starburst shaped, positioned within an inflated outer balloon 1361, creates patterned points of contact 1362c where the inner balloon 1363 contacts the outer balloon 1361. These points of contact 1362c create ablation zones for the transfer of ablative energy from the inner balloon through the outer balloon and to the target tissue. Gaps or openings 13620 are created where the irregularly shaped inner balloon 1363 does not contact the outer balloon 1361. Ablative energy is not transferred or transferred less at these openings 13620, allowing for blood to flow through these openings and also resulting in non-circumferential ablation.

FIG. 14 illustrates a side perspective view of an inner balloon 1404 configuration in a shape of a linear cylindrical tube 1406 with linear tubular extensions 1408, in accordance with some embodiments of the present specification. FIG. 15 illustrates a front view of inner balloon 1404. At least four tubular extensions 1408 extend over and above surface of the central linear cylindrical tube 1406 of diameter D1, so that surfaces of tubular volumes 1408 approximate the inner circumference of outer balloon 1402 when inner balloon 1404 is positioned centrally in an expanded state inside outer balloon 1402, such that the central longitudinal axis of inner balloon 1404 and outer balloon 1402 coincide. In embodiments, D1 is 4 mm. Each volume 1408 extends parallel to the central longitudinal axis of inner balloon 1404 and outer balloon 1402. In the pictured embodiment, each volume 1408 is equally spaced at a 90° angle from the other two adjacent volumes 1408, so that they cover the circumference of cylindrical tube 1406 in parallel and equidistant linear formations. In other embodiments, the inner balloon has fewer or more than four tubular extensions 1408 to create fewer or more ablation zones. An inner volume of inner balloon 1404 remains contiguous, therefore defining a starburst shape. In the embodiment pictured in FIG. 14B, hot or ablation zones 1403 are created at the contact points of the inner balloon 1404 with the outer balloon 1402. Cool or non-ablation zones 1405 are created where the outer balloon touches the target tissue but is not in contact with the inner balloon 1404.

Starburst shape of balloon 1404 extends at its two ends into tube-like structures or necks 1410 and 1412, which help in keeping the catheter in place when it is passed through the outer balloon. In one embodiment, the total length of balloon 1404 including the tubular necks or structures 1410, 1412 is in a range of 15 mm to 105 mm, and more preferably 25 mm to 60 mm, while the length of the starburst portion is in a range from 20 to 50 mm.

Similar to configurations of FIGS. 14 and 15, additional embodiments of inner balloon are possible, with diameter D of the linear cylindrical tube ranging from 2 to 6 mm, and the numbers of equidistant parallel linear extension tubular volumes on the surface of the linear cylindrical tube ranging from two to six or more. In embodiments, extensions 1408 and 1508 can have radii ranging from 1 to 3 mm. FIG. 16 illustrates a front view of another exemplary configuration of inner balloon 1604, with diameter D3 corresponding to the diameter of a linear cylindrical tube 1606, in accordance with an embodiment of the present specification. Embodiments of inner balloon 1604 includes three parallel tubular extensions 1608 extending linearly along the surface of linear cylindrical tube 1606. In embodiments, diameter D3 ranges from 3 to 20 mm. In embodiments, the radius of each of extensions 1608 ranges from 1 to 3 mm.

FIG. 17A illustrates a front view of another embodiment of an inner balloon 1704 configuration, in accordance with an embodiment of the present specification. FIG. 17B illustrates a side perspective view of inner balloon 1704. FIG. 17C illustrates a side view of balloon 1704. Referring simultaneously to FIGS. 17A, 17B and 17C, which show inner ablation balloon 1704 in its expanded state, a linear cylindrical tube 1706 is at the core with at least four parallel tubular extensions 1708a extending over and above surface of the linear cylindrical tube 1706, so that surfaces of tubular volumes 1708a approximate the inner circumference of an outer balloon 1702 when inner balloon 1704 is positioned centrally in an expanded state inside outer balloon 1702, where the central longitudinal axis of inner balloon 1704 and outer balloon 1702 coincide. Further, referring to FIGS. 17B and 17C, it is seen that tubular volumes 1708a extend from a distal end of inner balloon 1704 to half or less than half the length of linear cylindrical tube 1706, and then, after a small gap 1710, another set of parallel tubular volumes 1708b extend over the remaining half length of linear cylindrical tube 1706. The length of gap 1706 is of the diameter of tube 1706 and ranges between 1 mm to 6 mm. Each volume 1708a and 1708b extends parallel to the central longitudinal axis of inner balloon 1704 and outer balloon 1702. Each set of volumes 1708a and 1708b is equally spaced at a 90° angle from the other two adjacent volumes in the corresponding set, so that they cover the circumference of cylindrical tube 1706 in parallel and equidistant linear formations. An inner volume of inner balloon 1704 remains contiguous, therefore defining a split starburst shape. In embodiments, the outer balloon has a proximal neck inner diameter ranging from 1.98 mm to 3.96 mm and a distal neck inner diameter ranging from 1.39 mm to 2.79 mm. In embodiments, the inner balloon has a proximal neck inner diameter ranging from 1.90 mm to 3.81 mm and a distal neck inner diameter ranging from 1.72 mm to 3.45 mm. In embodiments, the catheter shaft has an outer diameter ranging from 1.88 to 3.73 mm. In the embodiment pictured in FIG. 17A, hot or ablation zones 1703 are created at the contact points of the inner balloon 1704 with the outer balloon 1702. Cool or non-ablation zones 1705 are created where the outer balloon touches the target tissue but is not in contact with the inner balloon 1704.

In an embodiment, the outer balloon 1702 is eliminated and only the inner balloon 1704 is used for performing ablation. The inner balloon 1704 by design, does not provide a 360° contact of the artery/vein being ablated, thereby allowing residual blood flow through the artery's finest structure. In said embodiment, the temperature of the inner balloon 1704 is maintained below 75° C. (<65° C. in a preferred embodiment), thereby allowing a cool or ambient perfusate to provide thermal protection without the need for the second outer balloon, thus enabling a simpler and safer configuration of the ablation system.

FIG. 17D illustrates a front view of a catheter 1720 having a single balloon 1724, wherein the balloon 1724 is not enclosed within an outer balloon, in accordance with an embodiment of the present specification. FIG. 17E illustrates a side perspective view of the balloon 1724 shown in FIG. 17D. FIG. 17F illustrates a cross sectional view of the balloon 1724 shown in FIG. 17D. In an embodiment, the balloon 1724 is spirally shaped. The catheter 1720 includes a distal end with a guidewire port 1730 and a proximal end with a handle 1736. A catheter shaft 1732 extends between the distal end and proximal end of the catheter 1720. In embodiments, the balloon 1724 is positioned on the catheter shaft 1732 proximate a distal end of the catheter 1720. In embodiments, a diameter of the catheter shaft 1732 ranges from 70 cm-110 cm. In an embodiment, a heating element 1734 comprising RF electrodes is positioned within the balloon 1724 for generating heat within the balloon 1724, thereby shifting heat generation from the catheter shaft to the balloon 1724 itself.

FIG. 17G illustrates an ablation catheter 1740 have a spiral shaped inner balloon 1743 within an outer balloon 1741, in accordance with some embodiments of the present specification. In embodiments, the catheter 1740 includes a handle 1745 with an inflation port 1746 at its proximal end. The catheter 1740 includes a catheter shaft having a proximal portion 1751 and a distal portion 1753 with a transition portion between 1752. In some embodiments, the proximal portion 1751 comprises a metallic hypotube. In embodiments, the distal portion 1753 includes an inner shaft for a guidewire and an outer shaft for inflating one or more of the balloons. In embodiments, the catheter includes a stiffening wire 1758 and a RX-Port 1749 and further includes radio-opaque markers 1749 on one or more of the balloons. In embodiments, the inner balloon 1743 has a spiral shaped and the contact portions of the inner balloon 1743 with the outer balloon create ablation zones for the transfer of ablative energy from the inner balloon, through the outer balloon, and to the target tissue. In embodiments, the catheter 1740 has the following dimensions as per Table 3 below.

TABLE 3

| Dimension | Range (mm) | Description |
|---|---|---|
| (D_{E}) | 0.40-0.50 | Lesion entry diameter (distal tip profile) |
| (D_{O}) | 0.81-0.93 | Outer shaft diameter |
| (D_{H}) | 0.59-0.69 | Hypotube diameter |
| (Dᵢ) | 0.53-0.56 | Inner shaft diameter (guidewire lumen) |
| (D_{B}) | 0.90-1.20 | Balloon outer diameter (crossing profile) |

The guidewire shaft length (L_{GW}) (RX port to distal tip) ranges between 230 mm and 250 mm.

The transition length, defined as the segment between the proximal end of the hypotube and the RX port, ranges from 71 mm to 115 mm.

The total catheter length (Lₜₒₜₐₗ) ranges approximately 1350 mm or greater, consistent with standard interventional cardiology catheter configurations.

Definition of dimensions:
(L_{GW}): Length of the guidewire-compatible inner shaft, measured from the rapid-exchange (RX) port to the distal tip of the catheter.
(Lₜₒₜₐₗ): Total longitudinal length of the catheter assembly, extending from the proximal handle to the distal tip.
(D_{E}): Lesion entry diameter, defined as the outer diameter at the distal tip of the catheter, typically representing the smallest crossing profile.
(D_{O}): Outer diameter of the outer shaft, which serves as the primary inflation lumen or structural conduit.
(D_{H}): Outer diameter of the hypotube, typically located in the proximal catheter shaft.
(D_{B}): Outer diameter of the balloon, measured in either the folded or inflated state, as applicable to the crossing profile.

These dimensions may be varied based on intended clinical use (e.g., coronary vs. peripheral vasculature), balloon compliance, material selection, or other structural considerations. The ranges provided herein are non-limiting and serve to illustrate representative embodiments of the inventive catheter system. Dimensions of inner spiral balloon and outer cylindrical balloon can be picked from the dimensions in the specifications.

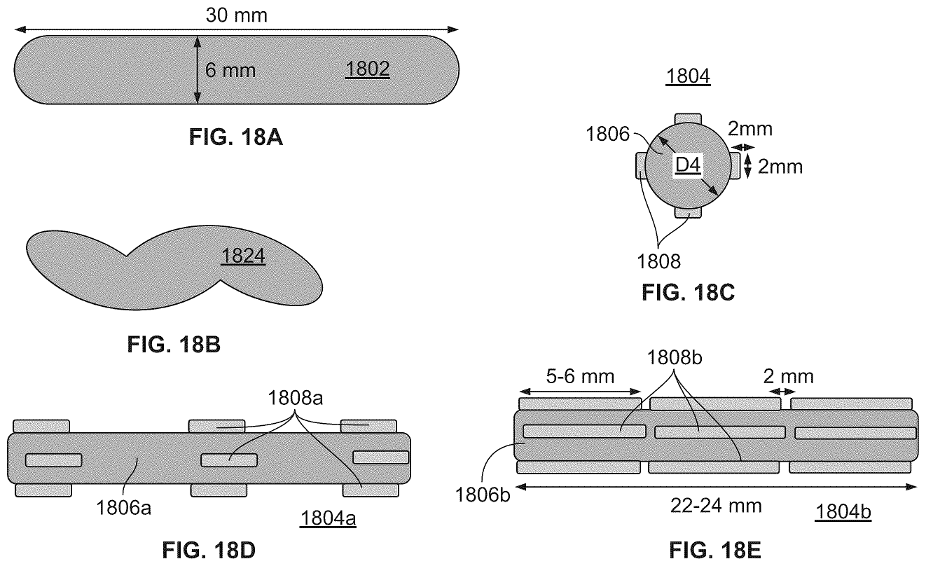

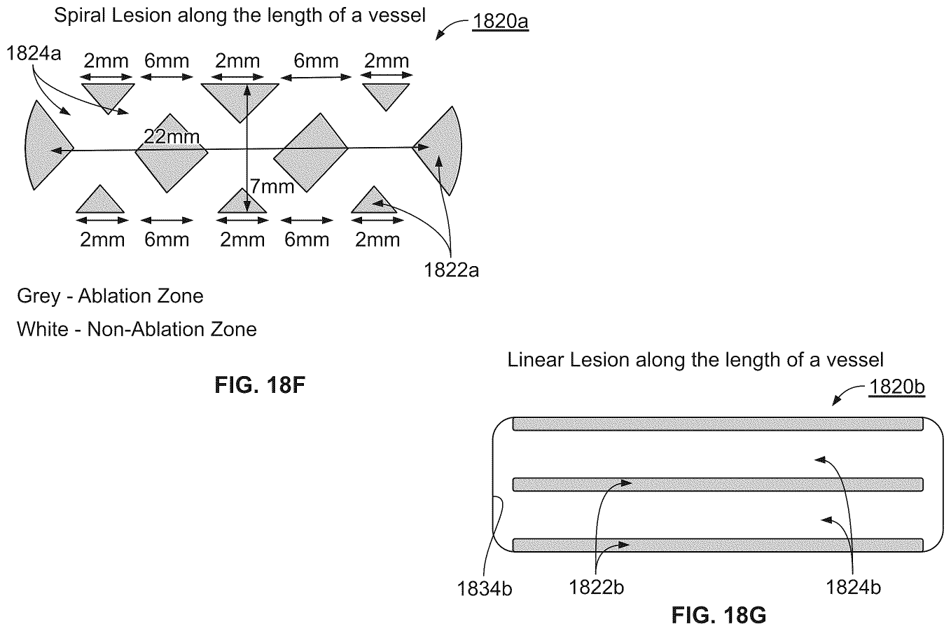

FIG. 18A illustrates an exemplary outer balloon 1802 in its expanded state, in accordance with some embodiments of the present specification. FIG. 18B illustrates an inner balloon 1824 with an irregular or spiral shape in the expanded state, in accordance with some embodiments of the present specification. FIG. 18C illustrates a cross-sectional view of an inner ablation balloon 1804 in its expanded state for ablation with outer balloon 1802 of FIG. 18A. The diameter D4 of linear cylindrical tube 1806 forming a base for inner ablation balloon 1804 can range from 5 to 6 mm. Parallel linear rectangular tubular volumes 1808 are positioned at equal radial spaces from each other around the circumference of tube 1806. FIG. 18D illustrates a side view of a first embodiment 1804a of inner ablation balloon 1804. FIG. 18E illustrates a side view of a second embodiment 1804b of inner ablation balloon 1804. In the first embodiment 1804a, parallel linear rectangular tubular volumes 1808a are split in at least three sections along the inner balloon. In embodiments, when the balloons are inflated, a total area of the tubular volumes 1808a configured to contact an inner surface of an outer balloon comprises <50% of a surface area of the outer surface of the inner balloon. In the second embodiment 1804b, parallel linear rectangular tubular volumes 1808b are split in at least three sections such that length of each volume 1808b is in a range of 5-6 mm and the length of a space or gap 1810b between two linear volumes 1808b is in a range of 1-3 mm. Overall length of inner ablation balloon 1804a and 1804b ranges from 22 to 24 mm.

In various embodiments, when inflated, the one or more balloons may have a shape such as, but not limited to, conical, square, spherical, elliptical, conical-square, long conical-square, conical-spherical, long spherical, tapered, dog bone, stepped, offset and conical-offset.

Again, in various embodiments, an end (distal and/or proximal) of the one or more balloons may have a shape such as, but not limited to, conical sharp corner, conical radius corner, square end, spherical end and offset neck.

FIG. 18F illustrates an exemplary spiral ablation geometry 1100a achieved using the balloon configurations of FIGS. 13A-13C and 18B, in accordance with some embodiments of the present specification. Geometry 1820a is achieved from left to right along the length of renal artery. Grey areas 1822a of geometry 1820a indicate the area of ablation (where the spirals of the inner balloon of FIG. 18B contact the outer balloon), and the non-grey areas 1124a represent areas of non-ablation (where the inner and outer balloons do not contact). The actual ablation geometry depends on the inner surface of the renal artery, shape and geometry of the inner ablation balloon and outer balloon, and shape of the perforated layer. Geometry 1820a is achieved with a specific shape and geometry of the inner ablation balloon, which leaves non-ablation zones of approximately 2 mm spaced between ablation zones of approximately 6 mm, along the length of the artery. In some embodiments, the diameter of the circumferential non-ablation and ablation zones is approximately 7 mm and/or is the same as the diameter of the renal artery. Total length of the non-ablation and ablation zones can extend to approximately 22 mm.

FIG. 18G illustrates an exemplary linear ablation geometry 1820b achieved using the balloon configurations shown in FIGS. 14A-17C, 18A, and 18C-18E, in accordance with some embodiments of the present specification. Geometry 1820b is achieved longitudinally along the length of the renal artery in the form of parallel ablation zones 1122b (where the linear tubular extensions of the inner balloon contact the outer balloon) spaced from each other with parallel non-ablation zones (where the inner and outer balloons do not contact) positioned between. A line 1834b illustrates possible border of an outer balloon within which ablation zones 1822b and non-ablation zones 1824b are formed by contact or lack of contact with the inner ablation balloon.

### Catheter Shaft

In various embodiments, the catheter bodies of the present specification comprise an extruded shaft with multiple lumens within. In other embodiments, the catheter bodies of the present specification comprise a single lumen with a plurality of bundled liners within said single lumen. The bundled liners are configured to function as separate independent lumens for the various functions of the catheter, including inflating agent delivery and suction and ablative agent delivery and suction. In various embodiments, each of the plurality of bundled liners has a different diameter. The separate lumens provide easier identification of each lumen, greater independence for the inner balloon, and air gaps between the bundled liners for insulation. In one embodiment, a vapor delivery lumen further includes an insulation liner at its distal end. In one embodiment, the insulation liner comprises a braided polyimide/Pebax shaft.

In some embodiments, the catheter is configured to have a temperature differential between an exterior surface of the catheter and an interior surface of the lumen where heated vapor is generated that is no less than 40 degrees Celsius. More specifically, the exterior surface of the catheter should be less than 60 degrees Celsius and preferably less than 45 degrees Celsius. In some embodiments, the catheter shaft is insulated with a guide sheath and a constant saline flow. The insulation prevents conduction from increasing shaft temperatures during steam generation.

### Circumference and Depth of Ablation

The ablation systems and methods of the present specification are configured to provide ablation within a vessel wall to achieve ablation of a cumulative circumference of less than 50%, less than 35%, or less than 20% at any cross-section of the vessel. In embodiments, a cumulative length of the ablation is greater than the cumulative circumference of the ablation. Additionally, the ablation systems and methods of the present specification are configured to provide ablation within a vessel wall to achieve depth of ablation that is transmural in at least more than 30%, more than 50%, or more than 70% of the ablation area.

The above examples are merely illustrative of the many applications of the system of the present invention. Although only a few embodiments of the present invention have been described herein, it should be understood that the present invention might be embodied in many other specific forms without departing from the spirit or scope of the invention. Therefore, the present examples and embodiments are to be considered as illustrative and not restrictive, and the invention may be modified within the scope of the appended claims.

What is claimed is:

1. A method of ablating a target tissue, the method comprising:
positioning a catheter proximate the target tissue of a patient, wherein the catheter comprises an elongate body having a lumen, a proximal end and a distal end and wherein an outer balloon, a perforated layer and an inner balloon are positioned at the distal end such that the inner balloon is positioned within the outer balloon and the perforated layer intervenes between the inner balloon and the outer balloon, wherein the perforated layer comprises a contiguous sheet with non-contiguous gaps;
inflating the outer balloon with a first fluid to increase a pressure of the outer balloon to a first outer balloon pressure;
expanding the perforated layer within the inflated outer balloon; and
infusing heated vapor into the inner balloon to increase a pressure of the inner balloon to a first inner balloon pressure, wherein infusing heated vapor into the inner balloon creates an ablation zone and wherein a surface area of the ablation zone is defined by a portion of the inner balloon contacting a portion of the outer balloon through the perforated layer to thereby allow for heat transfer from the heated vapor in the inner balloon through the ablation zone to the vascular tissue, wherein the ablation zone comprises a therapeutic ablation zone where a first portion of the inner balloon contacts a first portion of the outer balloon, and a sub-therapeutic zone where a second portion of the inner balloon contacts a second portion of the outer balloon through the contiguous sheet of the perforated layer.

2. The method of claim 1, wherein the target tissue is a renal artery, a portion of the renal artery, a renal vein, or a portion of the renal vein.

3. The method of claim 1, wherein the first pre-determined period of time is between 1 second and 5 minutes.

4. The method of claim 3, wherein the first outer balloon pressure is maintained for the first pre-determined period of time.

5. The method of claim 1, wherein the catheter comprises a plurality of electrodes positioned proximate the distal end and wherein the heated vapor is generated by directing saline through the lumen and over the plurality of electrodes.

6. The method of claim 1 wherein the first fluid is air or CO₂.

7. The method of claim 1, wherein the heated vapor comprises steam and wherein a temperature of the heated vapor is at least 100° C.

8. A system of ablating a target tissue, the system comprising:
a catheter, wherein the catheter comprises:
a proximal end;
a distal end;
an elongate body extending between the proximal end and the distal end;
a first lumen;
a second lumen;
at least one heating chamber positioned within the second lumen and configured to generate heated vapor from a fluid;
an outer balloon positioned at the distal end;
an inner balloon, wherein the inner balloon is positioned within the outer balloon; and
a perforated layer intervening between the inner balloon and the outer balloon, wherein the perforated layer comprises a contiguous sheet with non-contiguous gaps;
an RF generator electrically coupled to the at least one heating chamber;
an air pump in fluid communication with the outer balloon through the first lumen; and
a water pump in fluid communication with the inner balloon through the second lumen;
wherein infusing heated vapor into the inner balloon creates a therapeutic ablation zone where a first portion of the inner balloon contacts a first portion of the outer balloon through the non-contiguous gaps of the perforated layer, and a sub-therapeutic ablation zone where a second portion of the inner balloon contacts a second portion of the outer balloon through the contiguous sheet of the perforated layer.

9. The system of claim 8 wherein the outer balloon comprises a plurality of electrodes attached to an outer surface of the outer balloon.

10. The system of claim 9 wherein the plurality of electrodes are sensor electrodes configured to sense any of temperature, pressure, impedance or any signal that can be used to monitor or direct the treatment effect.

11. The system of claim 8 wherein the distal end comprises a plurality of infusion ports where the inner balloon is positioned.

12. The system of claim 11 wherein the plurality of infusion ports provide an exit for heated vapor generated by the at least one heating chamber into the inner balloon.

13. The system of claim 8, wherein the at least one heating chamber comprises a plurality of electrodes positioned proximate the distal end and wherein the heated vapor is generated by directing saline through a second lumen in the elongate body and over the plurality of electrodes.

14. The system of claim 8 wherein the first lumen communicates air or CO₂.

15. The system of claim 8, wherein the heated vapor comprises steam and wherein a temperature of the heated vapor is at least 100° C.

16. A catheter for ablating a target tissue, the catheter comprising:
an inflatable outer balloon at a distal end of the catheter;
an expandable inner balloon configured to expand when heated vapor is infused within the inner balloon, wherein the inner balloon is positioned within the outer balloon and the inner balloon is coaxial with the outer balloon; and
a perforated layer intervening between the inner balloon and the outer balloon, wherein the perforated layer comprises a contiguous sheet with non-contiguous gaps;
wherein infusing heated vapor into the inner balloon creates a therapeutic ablation zone where a first portion of the inner balloon contacts a first portion of the outer balloon through the non-contiguous gaps of the perforated layer, and a sub-therapeutic ablation zone where a second portion of the inner balloon contacts a second portion of the outer balloon through the contiguous sheet of the perforated layer.

17. The device of claim 16 wherein the outer balloon is inflated using air or CO₂.

18. The device of claim 16, wherein the heated vapor comprises steam and wherein a temperature of the heated vapor is at least 100° C.

19. The device of claim 16 wherein the inner balloon is smaller in size than the outer balloon by at least 10%.

20. The device of claim 16 wherein the inner balloon is movable along an axis of the inner balloon and the outer balloon.
